(19) United States
(12) Patent Application Publication
(10) Pub. No.: US 2020/0276333 A1
(43) Pub. Date: Sep. 3, 2020

(54) COMPOSITIONS AND METHODS FOR TREATING IDIOPATHIC OVERACTIVE BLADDER SYNDROME AND DETRUSOR OVERACTIVITY

(71) Applicant: ION CHANNEL INNOVATIONS, LLC, New York, NY (US)

(72) Inventors: Arnold MELMAN, Ardsley, NY (US); George CHRIST, Crozet, VA (US); Karl-Erik ANDERSSON, St Laurentiigatan (SE)

(73) Assignee: ION CHANNEL INNOVATIONS, LLC, New York, NY (US)

(21) Appl. No.: 16/612,286

(22) PCT Filed: May 14, 2018
(86) PCT No.: PCT/US2018/032574

§ 371 Date: Nov. 8, 2019

Related U.S. Application Data
(60) Provisional application No. 62/505,382, filed on May 12, 2017.

(51) Int. Cl. A61K 48/00; C12N 15/85

(52) U.S. Cl. CPC A61K 48/005; C12N 2830/50; C12N 15/85; A61K 48/0075

(57) ABSTRACT

The present invention provides methods of alleviating one or more signs or symptoms of smooth muscle diseases. Compositions of the disclosure may include a plasmid vector containing a nucleic acid that encodes a Maxi-K channel peptide. Compositions of the disclosure may be administered intradetrusorally to at least two or more sites at a single unit dose.

### RELATED APPLICATIONS

This application claims the benefit of provisional application U.S. Ser. No. 62/505,382, filed on May 12, 2017, the contents of which are herein incorporated by reference in their entirety.

### FIELD OF THE INVENTION

The present invention relates generally to the field of medical therapies to improve one or more symptoms related to smooth muscle dysfunction. In particular, smooth muscle dysfunction of the bladder.

### INCORPORATION OF SEQUENCE LISTING

The contents of the text file named IONC-002-001 WO_SeqList.txt, which was created on May 11, 2018 and is 30 KB in size, are hereby incorporated by reference in their entireties.

### BACKGROUND OF THE INVENTION

Abnormal bladder function is a common problem which significantly affects the quality of life of millions of men and women in the United States. Many common diseases (e.g., BHP, diabetes mellitus, multiple sclerosis, and stroke) alter normal bladder function. Significant untoward changes in bladder function are also a normal result of advancing age. There are two principal clinical manifestations of altered bladder physiology: the atonic bladder and the hyperreflexic bladder. The atonic bladder or detrusor underactivity has diminished capacity to empty its urine contents because of ineffective contractility of the detrusor smooth muscle (the outer smooth muscle of the bladder wall). In the atonic or underactive state, diminished smooth muscle contractility is implicated in the etiology of bladder dysfunction. Thus, it is not surprising that pharmacological modulation of smooth muscle tone is insufficient to correct the underlying problem. In fact, the prevailing method for treating this condition uses clean intermittent catheterization; this is a successful means of preventing chronic urinary tract infection, pyelonephritis, and eventual renal failure. As such, treatment of the atonic bladder ameliorates the symptoms of disease, but does not correct the underlying cause.

Conversely, the hyperreflexic, uninhibited, or bladder that exhibits detrusor overactivity contracts spontaneously during the filing of the bladder; this may result in urinary frequency, urinary urgency and urge incontinence, where the individual is unable to control the passage of urine. The hyperreflexic bladder is a more difficult problem to treat. Medications that have been used to treat this condition are usually only partially effective, and have severe side effects that limit the patient's use and enthusiasm. The currently-accepted treatment options (e.g., oxybutynin and tolteradine) are largely nonspecific, and most frequently involve blockade of the muscarinic-receptor pathways and/or the calcium channels on the bladder myocytes. Given the central importance of these two pathways in the cellular functioning of many organ systems in the body, such therapeutic strategies are not only crude methods for modulating bladder smooth muscle tone; rather, because of their very mechanism(s) of action, they are also virtually guaranteed to have significant and undesirable systemic effects. Accordingly, there is a great need for improved treatment options for bladder dysfunction.

Despite multiple attempts to develop a cure or treatment for diseases caused by altered smooth muscle tone, current therapies are inadequate because they provide limited efficacy and/or significant side effects. Thus, there is a long-felt need in the art for a pharmaceutical and/or medical intervention to address the underlying cause of altered smooth muscle tone by increasing efficacy with minimal side effects.

### SUMMARY OF THE INVENTION

The invention provides methods of treating or alleviating a sign or symptom of overactive bladder syndrome or detrusor overactivity in a human subject by administering intradetrusorally to at least two or more sites a unit dose of a composition comprising a vector having a promoter and a nucleic acid encoding a Maxi-K channel peptide. The promoter is for example, a smooth muscle promoter or a cytomegalovirus intermediate-early promoter. The unit dose is a single unit dose. Alternatively, two or more unit doses are administered at different times.

The unit dose is between about 5,000-50,000 mcg. For example, the unit dose is at least 10,000 mcg. Preferably, the unit dose is 16,000 mcg or 24,000 mcg.

The composition is administered at 5, 10, 15, 20 or more sites.

The sign or symptom is for example, frequency of micturition or urgency.

In some aspects the vector contains nucleic acid elements in the following order: a human cytomegalovirus intermediate-early promoter sequence, such as SEQ ID NO: 1; a T7 priming site sequence, such as SEQ ID NO: 2; a hSlo open reading frame sequence, such as SEQ ID NO: 7; a BGH polyadenylation signal sequence, such as SEQ ID NO: 3; a kanamycin resistance sequence, such as SEQ ID NO: 5 and a pUC origin of replication sequence, such as SEQ ID NO: 4. In certain aspects, the hSlo open reading frame sequence comprises a point mutation at position 1054 of SEQ ID NO: 7 resulting in a serine at position 352 of SEQ ID NO: 8.

The invention provides a vector, the vector comprising nucleic acid elements in the following order: a human cytomegalovirus intermediate-early promoter sequence such as SEQ ID NO: 1; a T7 priming site sequence such as SEQ ID NO: 2; a hSlo open reading frame sequence such as SEQ ID NO: 7; a BGH polyadenylation signal sequence such as SEQ ID NO: 3; a kanamycin resistance sequence such as SEQ ID NO: 5; and a pUC origin of replication sequence such as SEQ ID NO: 4. In some aspects of the vector, the hSlo open reading frame sequence has a single point mutation at nucleotide position 1054 of SEQ ID NO: 7, and said point mutation results in serine at position 352 of SEQ ID NO: 8. In some aspects, the vector comprises a plasmid, an adenoviral vector, an adeno-associated virus (AAV) vector, a retroviral vector or a liposome. In some aspects, the plasmid is pVAX.

The invention provides a pharmaceutical composition comprising a plurality of the vector of the disclosure and a pharmaceutically acceptable diluent or carrier. In some aspects, the pharmaceutical composition is formulated for injection into smooth muscle. In some aspects, the plurality of the vector is combined with a 20-25% sucrose in saline solution.

In some aspects of the pharmaceutical composition of the disclosure, the unit dose is a single unit dose. In some aspects, the unit dose is between about 5,000-50,000 mcg. In some aspects, the unit dose is at least 10,000 mcg. In some aspects, the unit dose is 16,000 mcg or 24,000 mcg.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### BRIEF DESCRIPTION OF THE DRAWINGS

FIG. 1 A-D is a series of four bar graphs which show micturition parameters following 2 weeks of obstruction in the two treatment groups.

FIG. 2 A-C is a series of three graphs which show cystometric recordings following 2 weeks of obstruction in a control group (FIG. 2A), a vector only (pVAX) group (FIG. 2B), and a group treated with hSlo (FIG. 2C).

FIG. 3 shows three graphs of cystometric recordings in a rat given vector only (pVAX), and 300 and 1000 μg of pVAX/hSLO.

FIG. 4 is a bar graph which shows average number of copies of pVAX/hSLO vector in tissues of female rats after two injections of 1000 μg.

FIG. 5 is a diagram which shows injection sites of the pVAX/hSLO vector in human subjects.

FIG. 6 is a bar graph showing the change in mean number of voids per day over time by treatment in human subjects. Error bars represent standard error of the means (SEM).

FIG. 7 is a bar graph showing the change in mean urgency episodes over time by treatment in human subjects. Error bars represent standard error of the means (SEM).

FIG. 8 is a plasmid map of pVAX/hSLO.

### DETAILED DESCRIPTION OF THE INVENTION

The present invention provides methods of gene therapy for treating physiological dysfunctions of the bladder. Specifically, the invention is based upon the discovery that direct injection of a vector that contains the gene that expresses the human Maxi-K channel (hMaxi-K) into the smooth muscle of the bladder wall significantly alleviated the symptoms of overactive bladder and urinary incontinence in women. Specifically, participants received a total dose of either 16,000 mcg or 24,000 mcg of hMaxi-K administered as 20-30 intramuscular injections into the bladder. Participants were seen 8 times within a 24-week period and a follow up at 18 months. The average diary data collected 7 days prior to each visit revealed statically significant reduction of voids per day as well as the mean number of urgency episodes per day for those participants receiving hMaxi-K compared to placebo.

The MaxiK channel (also known as the BK channel) provides an efflux pathway for potassium ions from the cell, allowing relaxation of smooth muscle by inhibition of the voltage sensitive Ca²⁺ channel, and thereby effecting normalization of organ function by reducing pathological heightened smooth muscle tone. The terms “MaxiK channel” and “BK channel” are used interchangeably herein.

Structurally, MaxiK channels are composed of alpha and beta subunits. Four alpha subunits form the pore of the channel, and these alpha subunits are encoded by a single Slo1 gene (also called hSlo, hSlo and potassium calcium-activated channel subfamily M alpha 1, or KCNM1). There are four beta subunits which can modulate MaxiK channel function. Each beta subunit has distinct tissue specific expression and modulatory functions, with the beta-1 subunit (potassium calcium-activated channel subfamily M regulator beta subunit 1, or KCNMB1) primarily expressed in smooth muscle cells.

Strategic clusters of MaxiK channels in close proximity to the ryanodine-sensitive calcium stores of the underlying sarcoplasmic reticulum provide an important mechanism for the local modulation of calcium signals (i.e., sparks) and membrane potential in diverse smooth muscle, including urinary bladder. The increase in the intracellular calcium level increases the open probability of the MaxiK channel, thus increasing the outward movement of K⁺ through the calcium sensitive MaxiK channel. The efflux of K⁺ causes a net movement of positive charge out of the cell, making the cell interior more negatively charged with respect to the outside. This has two major effects. First, the increased membrane potential ensures that the calcium channel spends more time closed than open. Second, because the calcium channel is more likely to be closed, there is a decreased net flux of Ca²⁺ into the cell and a corresponding reduction in the free intracellular calcium levels. The reduced intracellular calcium promotes smooth muscle relaxation. The major implication of having more MaxiK channels in the cell membrane, therefore, is that it should lead to enhanced smooth muscle cell relaxation to any given stimulus for relaxation.

Increased intercellular communication among detrusor myocytes occurs in both animal models of partial urethral obstruction (PUO) and humans with detrusor overactivity (DO). With respect to increased intercellular communication, the impact of increased calcium signaling may be augmented when compared to a normal bladder with potentially lower levels of intercellular coupling. This increased calcium signaling contributes, at least in part, to the “non-voiding contractions” observed in the PUO rat model. However, if there were a parallel increase in MaxiK channel expression (for example, as a result of over-expression of a MaxiK channel encoding transgene of a composition or method of the disclosure), then presumably the resultant recombinant and/or transgenic MaxiK channels expressed by these transfected cells may “short circuit” abnormally increased calcium signals. This prevents further spread through gap junctions, and thus, prevents sufficient augmentation of abnormal and increased calcium signaling (by, for example, non-transfected myocyte recruitment) to mitigate abnormal contractile responses. The reduction of abnormal contractile responses in individual cells or groups of cells, by over-expression of a MaxiK channel encoding transgene of a composition or method of the disclosure eliminates or ameliorates the non-voiding contractions characteristic of DO, the clinical correlate of urgency. Conversely, because the involvement of spinal reflexes in the micturition response produces coordinated detrusor contractions well in excess of the abnormally increased calcium signaling associated with DO, MaxiK transgene over-expression may effectively reduce or inhibit the weaker abnormally increased calcium signal that contributes to DO (as measured in an animal model as a decrease in IMP (intermicturition pressure) or SA (spontaneous activity compared to control levels), without significantly or detectably affecting the more robust micturition contraction response.

Aging and disease can result in changes in the expression of the final product of the hSlo gene, the gene that expresses the α-subunit of the large conductance Ca2+-activated, voltage sensitive potassium (BKα) channel. Those changes result in reduced organ-specific physiological modification of the tone of the smooth muscle that comprises the organ. The effect is heightened tone of the smooth muscle cells in the organs that cause human diseases such as erectile dysfunction (ED) in the penis, urinary urgency, frequency, nocturia, and incontinence in the bladder (e.g. over active bladder (OAB) syndrome), asthma in the lungs, irritable bowel in the colon, glaucoma in the eyes and bladder outlet obstruction in the prostate.

### Methods of the Invention

The present invention provides a method of gene therapy for treating physiological dysfunctions of smooth muscle. Specifically, the methods of the invention are used to treat or alleviate a symptom of overactive bladder (OAB) syndrome or detrusor overactivity.

OAB syndrome is characterized by a group of symptoms that include, but are not limited to, urinary urgency, frequency, nocturia and incontinence. OAB is subdivided into idiopathic OAB and neurogenic OAB

Detrusor overactivity is defined as a urodynamic observation characterized by involuntary detrusor contractions during the filling phase that may be spontaneous or provoked. Detrusor overactivity is subdivided into idiopathic detrusor overactivity and neurogenic detrusor overactivity.

The compositions and methods of the disclosure provide for the delivery of a nucleic acid encoding hMaxi-K to cells in a human subject or patient in need thereof. In some cases, delivery of the nucleic acid may be referred to as gene therapy.

The composition and methods of the disclosure provide for any suitable method for delivery of the hMaxi-K nucleic acid or mutant thereof. In some cases, delivery of the nucleic acid may be performed using any suitable “vector” (sometimes also referred to as “gene delivery” or “gene transfer” vehicle). Vector, delivery vehicle, gene delivery vehicle or gene transfer vehicle, may refer to any suitable macromolecule or complex of molecules comprising a polynucleotide to be delivered to a target cell. In some cases, a target cell may be any cell to which the nucleic acid or gene is delivered. The polynucleotide to be delivered may comprise a coding sequence of interest in gene therapy, such as the hSlo gene.

The hSlo gene is introduced into a smooth muscle cell of the bladder by direct injection into the detrusor muscle.

For example, suitable vectors may include but are not limited to, viral vectors such as adenoviruses, adeno-associated viruses (AAV), and retroviruses, liposomes, other lipid-containing complexes, and other macromolecular complexes capable of mediating delivery of a polynucleotide to a target cell.

Alternatively, the hSlo gene is transferred into the smooth muscle cells by naked DNA transfer, using a mammalian vector. “Naked DNA” is herein defined as DNA contained in a non-viral vector. The DNA sequence may be combined with a sterile aqueous solution, which is preferably isotonic with the blood of the recipient. Such a solution may be prepared by suspending the DNA in water containing physiologically-compatible substances (such as sodium chloride, glycine, and the like), maintaining a buffered pH compatible with physiological conditions, and rendering the solution sterile. In a preferred embodiment of the invention, the DNA is combined with a 20-25% sucrose-in-saline solution, (e.g. phosphate buffered saline) in preparation for introduction into a smooth muscle cell.

As described herein, nucleic acids may refer to polynucleotides. Nucleic acid and polynucleotide may be used interchangeably. In some cases nucleic acids may comprise DNA or RNA. In some aspects, nucleic acids may include DNA or RNA for the expression of Maxi-K. In some aspects RNA nucleic acids may include but are not limited to a transcript of a gene of interest (e.g. Slo), introns, untranslated regions, termination sequences and the like. In other cases, DNA nucleic acids may include but are not limited to sequences such as hybrid promoter gene sequences, strong constitutive promoter sequences, the gene of interest (e.g. Slo), untranslated regions, termination sequences and the like. In some cases, a combination of DNA and RNA may be used.

As described in the disclosure herein, the term “expression construct” is meant to include any type of genetic construct containing a nucleic acid or polynucleotide coding for gene products in which part or all of the nucleic acid encoding sequence is capable of being transcribed. The transcript may be translated into a protein. In some aspects it may be partially translated or not translated. In certain aspects, expression includes both transcription of a gene and translation of mRNA into a gene product. In other aspects, expression only includes transcription of the nucleic acid encoding genes of interest.

The nucleic acid may be measured as the quantity of nucleic acid. Generally, any suitable amount of nucleic acid may be used with the compositions and methods of this disclosure. In some cases, nucleic acid may be at least about 1 pg, 10 pg, 100 pg, 1 pg, 10 pg, 100 pg, 200 pg, 300 pg, 400 pg, 500 pg, 600 pg, 700 pg, 800 pg, 900 pg, 1 μg, 10 μg, 100 μg, 200 μg, 300 μg, 400 μg, 500 μg, 600 μg, 700 μg, 800 μg, 900 μg, 1 ng, 10 ng, 100 ng, 200 ng, 300 ng, 400 ng, 500 ng, 600 ng, 700 ng, 800 ng, 900 ng, 1 mg, 10 mg, 100 mg, 200 mg, 300 mg, 400 mg, 500 mg, 600 mg, 700 mg, 800 mg, 900 mg 1 g, 2 g, 3 g, 4 g, or 5 g. In some cases, nucleic acid may be at most about 1 pg, 10 pg, 100 pg, 1 pg, 10 pg, 100 pg, 200 pg, 300 pg, 400 pg, 500 pg, 600 pg, 700 pg, 800 pg, 900 pg, 1 μg, 10 μg, 100 μg, 200 μg, 300 μg, 400 μg, 500 μg, 600 μg, 700 μg, 800 μg, 900 μg, 1 ng, 10 ng, 100 ng, 200 ng, 300 ng, 400 ng, 500 ng, 600 ng, 700 ng, 800 ng, 900 ng, 1 mg, 10 mg, 100 mg, 200 mg, 300 mg, 400 mg, 500 mg, 600 mg, 700 mg, 800 mg, 900 mg, 1 g, 2 g, 3 g, 4 g, or 5 g.

In some cases nucleic acid may be at least about 5000 mcg, 7500 mcg, 10,000 mcg, 12,500 mcg, 15,000 mcg, 16,000 mcg, 17,500 mcg, 20,000 mcg, 22,500 mcg, 24,000 mcg 25,000 mcg, 30,000 mcg, 35,000 mcg, 40,000 mcg, 45,000 mcg or 50,000 mcg.

As used herein mcg and μg are used interchangeably.

The present invention specifically provides a method of gene therapy wherein the MaxiK channel protein involved in the regulation of smooth muscle tone modulates relaxation of smooth muscle. These proteins will promote or enhance relaxation of smooth muscle, and will thus decrease smooth muscle tone. In particular, where smooth muscle tone is decreased in the bladder, bladder capacity will be increased.

Furthermore, the present invention specifically provides a method of regulating bladder smooth muscle tone in a subject, comprising the introduction, into bladder smooth muscle cells of the subject, of a DNA sequence encoding a protein involved in the regulation of smooth muscle tone, and expression in a sufficient number of bladder smooth muscle cells of the subject to enhance bladder relaxation in the subject. In this embodiment, the method of the present invention is used to alleviate a hyperreflexic bladder. A hyperreflexic bladder may result from a variety of disorders, including neurogenic and arteriogenic dysfunctions, as well as other conditions which cause incomplete relaxation or heightened contractility of the smooth muscle of the bladder. The subject may be animal or human, and is preferably human.

The recombinant vectors and plasmids of the present invention may also contain a nucleotide sequence encoding suitable regulatory elements, so as to effect expression of the vector construct in a suitable host cell. As used herein, “expression” refers to the ability of the vector to transcribe the inserted DNA sequence into mRNA so that synthesis of the protein encoded by the inserted nucleic acid can occur. Those skilled in the art will appreciate the following: (1) that a variety of enhancers and promoters are suitable for use in the constructs of the invention; and (2) that the constructs will contain the necessary start, termination, and control sequences for proper transcription and processing of the DNA sequence encoding a protein involved in the regulation of smooth muscle tone, upon introduction of the recombinant vector construct into a host cell.

The non-viral vectors provided by the present invention, for the expression in a smooth muscle cell of the DNA sequence encoding a protein involved in the regulation of smooth muscle tone, may comprise all or a portion of any of the following vectors known to one skilled in the art: pVax (Thermnno Fisher Scientific), pCMVβ (Invitrogen), pcDNA3 (Invitrogen), pET-3d (Novagen), pProEx-1 (Life Technologies), pFastBac 1 (Life Technologies), pSFV (Life Technologies), pcDNA2 (Invitrogen), pSL301 (Invitrogen), pSE280 (Invitrogen), pSE380 (Invitrogen), pSE420 (Invitrogen), pTrcHis A,B,C (Invitrogen), pRSET A,B,C (Invitrogen), pYES2 (Invitrogen), pAC360 (Invitrogen), pVL1392 and pV11392 (Invitrogen), pCDM8 (Invitrogen), pcDNA I (Invitrogen), pcDNA I(amp) (Invitrogen), pZeoSV (Invitrogen), pRc/CMV (Invitrogen), pRc/RSV (Invitrogen), pREP4 (Invitrogen), pREP7 (Invitrogen), pREP8 (Invitrogen), pREP9 (Invitrogen), pREP10 (Invitrogen), pCEP4 (Invitrogen), pEBVHis (Invitrogen), and λPop6. Other vectors would be apparent to one skilled in the art. Preferably the vector is pVax.

In some embodiments, the pVax vector sequence comprises a sequence of:

| (SEQ ID NO: 10) | |
|---|---|
| 1 | GACTCTTCGC GATGTACGGG CCAGATATAC GCGTTGACAT TGATTATTGA CTAGTTATTA |
| 61 | ATAGTAATCA ATTACGGGGT CATTAGTTCA TAGCCCATAT ATGGAGTTCC GCGTTACATA |
| 121 | ACTTACGGTA AATGGCCCGC CTGGCTGACC GCCCAACGAC CCCCGCCCAT TGACGTCAAT |
| 181 | AATGACGTAT GTTCCCATAG TAACGCCAAT AGGGACTTTC CATTGACGTC AATGGGTGGA |
| 241 | CTATTTACGG TAAACTGCCC ACTTGGCAGT ACATCAAGTG TATCATATGC CAAGTACGCC |
| 301 | CCCTATTGAC GTCAATGACG GTAAATGGCC CGCCTGGCAT TATGCCCAGT ACATGACCTT |
| 361 | ATGGGACTTT CCTACTTGGC AGTACATCTA CGTATTAGTC ATCGCTATTA CCATGGTGAT |
| 421 | GCGGTTTTGG CAGTACATCA ATGGGCGTGG ATAGCGGTTT GACTCACGGG GATTTCCAAG |
| 481 | TCTCCACCCC ATTGACGTCA ATGGGAGTTT GTTTTGGCAC CAAAATCAAC GGGACTTTCC |
| 541 | AAAATGTCGT AACAACTCCG CCCCATTGAC GCAAATGGGC GGTAGGCGTG TACGGTGGGA |
| 601 | GGTCTATATA AGCAGAGCTC TCTGGCTAAC TAGAGAACCC ACTGCTTACT GGCTTATCGA |
| 661 | AATTAATACG ACTCACTATA GGGAGACCCA AGCTGGCTAG CGTTTAAACT TAAGCTTGGT |
| 721 | ACCGAGCTCG GATCCACTAG TCCAGTGTGG TGGAATTCTG CAGATATCCA GCACAGTGGC |
| 781 | GGCCGCTCGA GTCTAGAGGG CCCGTTTAAA CCCGCTGATC AGCCTCGACT GTGCCTTCTA |
| 841 | GTTGCCAGCC ATCTGTTGTT TGCCCCTCCC CCGTGCCTTC CTTGACCCTG GAAGGTGCCA |
| 901 | CTCCCACTGT CCTTTCCTAA TAAAATGAGG AAATTGCATC GCATTGTCTG AGTAGGTGTC |
| 961 | ATTCTATTCT GGGGGGTGGG GTGGGGCAGG ACAGCAAGGG GGAGGATTGG GAAGACAATA |
| 1021 | GCAGGCATGC TGGGGATGCG GTGGGCTCTA TGGCTTCTAC TGGGCGGTTT TATGGACAGC |
| 1081 | AAGCGAACCG GAATTGCCAG CTGGGGCGCC CTCTGGTAAG GTTGGGAAGC CCTGCAAAGT |
| 1141 | AAACTGGATG GCTTTCTCGC CGCCAAGGAT CTGATGGCGC AGGGGATCAA GCTCTGATCA |
| 1201 | AGAGACAGGA TGAGGATCGT TTCGCATGAT TGAACAAGAT GGATTGCACG CAGGTTCTCC |
| 1261 | GGCCGCTTGG GTGGAGAGGC TATTCGGCTA TGACTGGGCA CAACAGACAA TCGGCTGCTC |
| 1321 | TGATGCCGCC GTGTTCCGGC TGTCAGCGCA GGGGCGCCCG GTTCTTTTTG TCAAGACCGA |
| 1381 | CCTGTCCGGT GCCCTGAATG AACTGCAAGA CGAGGCAGCG CGGCTATCGT GGCTGGCCAC |
| 1441 | GACGGGCGTT CCTTGCGCAG CTGTGCTCGA CGTTGTCACT GAAGCGGGAA GGGACTGGCT |
| 1501 | GCTATTGGGC GAAGTGCCGG GGCAGGATCT CCTGTCATCT CACCTTGCTC CTGCCGAGAA |
| 1561 | AGTATCCATC ATGGCTGATG CAATGCGGCG GCTGCATACG CTTGATCCGG CTACCTGCCC |
| 1621 | ATTCGACCAC CAAGCGAAAC ATCGCATCGA GCGAGCACGT ACTCGGATGG AAGCCGGTCT |
| 1681 | TGTCGATCAG GATGATCTGG ACGAAGAGCA TCAGGGGCTC GCGCCAGCCG AACTGTTCGC |
| 1741 | CAGGCTCAAG GCGAGCATGC CCGACGGCGA GGATCTCGTC GTGACCCATG GCGATGCCTG |
| 1801 | CTTGCCGAAT ATCATGGTGG AAAATGGCCG CTTTTCTGGA TTCATCGACT GTGGCCGGCT |
| 1861 | GGGTGTGGCG GACCGCTATC AGGACATAGC GTTGGCTACC CGTGATATTG CTGAAGAGCT |
| 1921 | TGGCGGCGAA TGGGCTGACC GCTTCCTCGT GCTTTACGGT ATCGCCGCTC CCGATTCGCA |
| 1981 | GCGCATCGCC TTCTATCGCC TTCTTGACGA GTTCTTCTGA ATTATTAACG CTTACAATTT |
| 2041 | CCTGATGCGG TATTTTCTCC TTACGCATCT GTGCGGTATT TCACACCGCA TACAGGTGGC |
| 2101 | ACTTTTCGGG GAAATGTGCG CGGAACCCCT ATTTGTTTAT TTTTCTAAAT ACATTCAAAT |
| 2161 | ATGTATCCGC TCATGAGACA ATAACCCTGA TAAATGCTTC AATAATAGCA CGTGCTAAAA |
| 2221 | CTTCATTTTT AATTTAAAAG GATCTAGGTG AAGATCCTTT TTGATAATCT CATGACCAAA |
| 2281 | ATCCCTTAAC GTGAGTTTTC GTTCCACTGA GCGTCAGACC CCGTAGAAAA GATCAAAGGA |
| 2341 | TCTTCTTGAG ATCCTTTTTT TCTGCGCGTA ATCTGCTGCT TGCAAACAAA AAAACCACCG |
| 2401 | CTACCAGCGG TGGTTTGTTT GCCGGATCAA GAGCTACCAA CTCTTTTTCC GAAGGTAACT |
| 2461 | GGCTTCAGCA GAGCGCAGAT ACCAAATACT GTCCTTCTAG TGTAGCCGTA GTTAGGCCAC |
| 2521 | CACTTCAAGA ACTCTGTAGC ACCGCCTACA TACCTCGCTC TGCTAATCCT GTTACCAGTG |
| 2581 | GCTGCTGCCA GTGGCGATAA GTCGTGTCTT ACCGGGTTGG ACTCAAGACG ATAGTTACCG |
| 2641 | GATAAGGCGC AGCGGTCGGG CTGAACGGGG GGTTCGTGCA CACAGCCCAG CTTGGAGCGA |
| 2701 | ACGACCTACA CCGAACTGAG ATACCTACAG CGTGAGCTAT GAGAAAGCGC CAGGCTTCCC |
| 2761 | GAAGGGAGAA AGGCGGACAG GTATCCGGTA AGCGGCAGGG TCGGAACAGG AGAGCGCACG |
| 2821 | AGGGAGCTTC CAGGGGGAAA CGCCTGGTAT CTTTATAGTC CTGTCGGGTT TCGCCACCTC |
| 2881 | TGACTTGAGC GTCGATTTTT GTGATGCTCG TCAGGGGGGC GGAGCCTATG GAAAAACGCC |
| 2941 | AGCAACGCGG CCTTTTTACG GTTCCTGGGC TTTTGCTGGC CTTTTGCTCA CATGTTCTT. |

In some embodiments, the pVAX sequence comprises a sequence with at least 95%, at least 96%, at least 97%, at least 98% or at least 99% identity to SEQ ID NO: 10. In some embodiments, the pVAX sequence comprises a substitution of G for A at position 2 of SEQ ID NO: 10, an additional G at position 5 of SEQ ID NO: 10, a substitution of T for C at position 1158 of SEQ ID NO: 10, a missing A at position 2092 of SEQ ID NO: 10, a substitution of T for C at position 2493 of SEQ ID NO: 10, or a combination thereof.

Promoters suitable for the present invention include, but are not limited to, constitutive promoters, tissue-specific promoters, and inducible promoters. In some embodiments the promoter is a smooth muscle promotor. In other embodiments the promotor is a muscle cell promotor. Preferably, the promotor is not an urothelium specific expression promotor.

In one embodiment of the invention, expression of the DNA sequence encoding a protein involved in the regulation of smooth muscle tone is controlled and affected by the particular vector into which the DNA sequence has been introduced. Some eukaryotic vectors have been engineered so that they are capable of expressing inserted nucleic acids to high levels within the host cell. Such vectors utilize one of a number of powerful promoters to direct the high level of expression. Eukaryotic vectors use promoter-enhancer sequences of viral genes, especially those of tumor viruses. This particular embodiment of the invention provides for regulation of expression of the DNA sequence encoding the protein, through the use of inducible promoters. Non-limiting examples of inducible promoters include metallothionine promoters and mouse mammary tumor virus promoters. Depending on the vector, expression of the DNA sequence in the smooth muscle cell would be induced by the addition of a specific compound at a certain point in the growth cycle of the cell. Other examples of promoters and enhancers effective for use in the recombinant vectors of the present invention include, but are not limited to, CMV (cytomegalovirus), SV40 (simian virus 40), HSV (herpes simplex virus), EBV (Epstein-Barr virus), retrovirus, adenoviral promoters and enhancers, and smooth-muscle-specific promoters and enhancers. An example of a smooth-muscle-specific promoter is SM22α. Exemplary smooth muscle promoters are described in U.S. Pat. No. 7,169,764, the contents of which are herein incorporated by reference in its entirety.

In preferred embodiments, the promotor is a SM22α promoter sequence and may include but is not limited to sequences such as:

(SEQ ID NO: 9)
gaattcagga cgtaatcagt ggctggaaag caagagctct
agaggagctc tgacccttcc ttcagatgcc acaaggaggt
gctggagttc tatgcaccaa cagccaggct ggctgtagtg
gattgagcgt ctgaggctgc acctctctgg gttctgggtg
agactgaccc tgcctgaggg ttctctcctt ccctctctct
ccctctccct ctccctctct ctgtttcctg aggtttccag
gattggggat gacaccacta aagccttacc ttttaagaag
ttgcattcag tgagtgtgtg cagatagggg cagaggagag
ctggttctgt ctccactgtg tttggtcttg tcagaccatc
aggtgtgata gcagttgtct ttaaccctaa ccctgagcct
tcccttccca agaccactga agctaggtgc aagataagtg
gggacccttt aggatctttc acgataagga ctattttgaa
gggagggagg gtgacactgt ttaccctagt gtctccagcc
ttgccaggcc ttaaacatcc gcccattgtc aaggggccag
ggttgacttg ctgctaaaca aggcactccc tagagaagca
gcataccata cctgtgggca ggatgaccca tgttctgcca
cgcacttggt aggccacttt gaacctcaat tttctcaact
gttaaatggg gtggtaactg ataaagggga acgtgaaagg
aaggcgtttg catagtgcct ggttgtgcag gtcaagacta
gttcccacca actcgatttt aaagccttgc aagaaggtgg
ccttgcaggt tcctttgtcg ggccaaactc tagaatgcct
ccccctttct agagcagacc caagtccggg taacaaggaa
gggtttcagg gtcctgccca ttcccggccg ccctcagcac
cgccccgccc cgacccccgc agcatctcca cagcttatta
tagcttaaac cctgcagcca actcctttct gggactcaga
agacatagca ggtactgaac gtctcacctg ctgaggtggt
cctagtcctc acccgctcta gcccgctaga agccttggaa
ctatctcata ccaggctgca cttgtttgtc ttctcattga
taaaaggttt aagcatgcag agaatgtctc cggctgcccc
cgacagactg ctccaacttg gtgtctttcc ccaaatatgg
agcctgtgtg gagtgagtgg ggcggcccgg ggtggtgagc
caagcagact tccatgggca gggaggggcg ccagcggacg
gcagaggggt gacatcactg cctaggcggc ctttaaaccc
ctcacccagc cggcgcccca gcccgtctgc cccagcccag
acaccgaagc tactctcctt ccagtccaca aacgaccaag
ccttgtaagt gcaagtcat.

In preferred embodiments, the promotor is a human cytomegalovirus intermediate-early promoter sequence and may include but is not limited to sequences such as:

(SEQ ID NO: 1)
CGTTACATAACTTACGGTAAATGGCCCGCCTGGCTGACCGCCCAACGACCC
CCGCCCATTGACGTCAATAATGACGTATGTTCCCATAGTAACGCCAATAGG
GACTTTCCATTGACGTCAATGGGTGGAGTATTTACGGTAAACTGCCCACTT
GGCAGTACATCAAGTGTATCATATGCCAAGTACGCCCCCTATTGACGTCAA
TGACGGTAAATGGCCCGCCTGGCATTATGCCCAGTACATGACCTTATGGGA
CTTTCCTACTTGGCAGTACATCTACGTATTAGTCATCGCTATTACCATGGT
GATGCGGTTTTGGCAGTACATCAATGGGCGTGGATAGCGGTTTGACTCACG
GGGATTTCCAAGTCTCCACCCCATTGACGTCAATGGGAGTTTGTTTTGGCA
CCAAAATCAACGGGACTTTCCAAAATGTCGTAACAACTCCGCCCCATTGAC
GCAAATGGGCGGTAGGCGTGTACGGTGGGAGGTCTATATAAGCAGAGCT.

In some aspects, a T7 priming site may be included such as, but is not limited to, sequences such as TAATACGACTCACTATAGGG SEQ ID NO: 2.

In some aspects, the recombinant virus and/or plasmid used to express a DNA sequence or protein of the disclosure comprises a polyA (polyadenylation) sequence, such as those provided herein (e.g., BGH polyA sequence.). Generally, any suitable polyA sequence may be used for the desired expression of the transgene For example, in some cases, the present disclosure provides for a sequence comprising BGH polyA sequence, or portion of a BGH polyA sequence. In some cases, the present disclosure provides for polyA sequences comprising a combination of one or more polyA sequences or sequence elements. In some cases, no polyA sequence is used. In some cases one or more polyA sequences may be referred to as untranslated regions (UTRs), 3′ UTRs, or termination sequences.

A polyA sequence may comprise a length of 1-10 bp, 10-20 bp, 20-50 bp, 50-100 bp, 100-500 bp, 500 bp-1 Kb, 1 Kb-2 Kb, 2 Kb-3 Kb, 3 Kb-4 Kb, 4 Kb-5 Kb, 5 Kb-6 Kb, 6 Kb-7 Kb, 7 Kb-8 Kb, 8 Kb-9 Kb, and 9 Kb-10 Kb in length. A polyA sequence may comprise a length of at least 1 bp, 2 bp, 3 bp, 4 bp, 5 bp, 6 bp, 7 bp, 8 bp, 9 bp, 10 bp, 20 bp, 30 bp, 40 bp, 50 bp, 60 bp, 70 bp, 80 bp, 90 bp, 100 bp, 200 bp, 300 bp, 400 bp, 500 bp, 600 bp, 700 bp, 800 bp, 900 bp, 1 Kb, 2 Kb, 3 Kb, 4 Kb, 5 Kb, 6 Kb, 7 Kb, 8 Kb, 9 Kb, and 10 Kb in length. A polyA sequence may comprise a length of at most 1 bp, 2 bp, 3 bp, 4 bp, 5 bp, 6 bp, 7 bp, 8 bp, 9 bp, 10 bp, 20 bp, 30 bp, 40 bp, 50 bp, 60 bp, 70 bp, 80 bp, 90 bp, 100 bp, 200 bp, 300 bp, 400 bp, 500 bp, 600 bp, 700 bp, 800 bp, 900 bp, 1 Kb, 2 Kb, 3 Kb, 4 Kb, 5 Kb, 6 Kb, 7 Kb, 8 Kb, 9 Kb, and 10 Kb in length.

In some cases, a BGH polyA may include but is not limited to sequences such as:

(SEQ ID NO: 3)
agcctcgact gtgccttcta gttgccagcc atctgttgtt
tgcccctccc ccgtgccttc cttgaccctg gaaggtgcca
ctcccactgt cctttcctaa taaaatgagg aaattgcatc
gcattgtctg agtaggtgtc attctattct ggggggtggg
gtggggcagg acagcaaggg ggaggattgg gaagacaata
gcaggcatgc tggggatgcg gtgggctcta gtgggctct.

In some cases, polyA sequences may be optimized for various parameters affecting protein expression, including but not limited to mRNA half-life of the transgene in the cell, stability of the mRNA of the transgene or transcriptional regulation. For example, polyA sequences may be altered to increase mRNA transcription of the transgene, which may result in increased protein expression. In some cases, the polyA sequences may be altered to decrease the half-life of the mRNA transcript of the transgene, which may result in decreased protein expression.

In some aspects, the vector, comprises a sequence encoding a replication origin sequence, such as those provided herein. Origin of replication sequences, generally provide sequence useful for propagating a plasmid/vector.

In some cases, a pUC origin of replication sequence may be include but is not limited to sequences such as:

(SEQ ID NO: 4)
ccgtagaaaa gatcaaagga tcttcttgag atcctttttt
tctgcgcgta atctgctgct tgcaaacaaa aaaaccaccg
ctaccagcgg tggtttgttt gccggatcaa gagctaccaa
ctctttttcc gaaggtaact ggcttcagca gagcgcagat
accaaatact gtccttctag tgtagccgta gttaggccac
cacttcaaga actctgtagc accgcctaca tacctcgctc
tgctaatcct gttaccagtg gctgctgcca gtggcgataa
gtcgtgtctt accgggttgg actcaagacg atagttaccg
gataaggcgc agcggtcggg ctgaacgggg ggttcgtgca
cacagcccag cttggagcga acgacctaca ccgaactgag
atacctacag cgtgagctat gagaaagcgc cacgcttccc
gaagggagaa aggcggacag gtatccggta agcggcaggg
tcggaacagg agagcgcacg agggagcttc cagggggaaa
cgcctggtat ctttatagtc ctgtcgggtt tcgccacctc
tgacttgagc gtcgattttt gtgatgctcg tcaggggggc
ggagcctatg gaaaaacgcc agcaacgcgg cctttttacg
gttcctgggc ttttgctggc cttttgctca catgttctt.

The vector may also comprise a selectable marker. Selectable markers can be positive, negative or bifunctional. Positive selectable markers allow selection for cells carrying the marker, whereas negative selectable markers allow cells carrying the marker to be selectively eliminated. A variety of such marker genes have been described, including bifunctional (i.e., positive/negative) markers (see, e.g., Lupton, S., WO 92/08796, published May 29, 1992; and Lupton, S., WO 94/28143, published Dec. 8, 1994). Examples of negative selectable markers may include the inclusion of resistance genes to antibiotics, such as ampicillin or kanamycin. Such marker genes can provide an added measure of control that can be advantageous in gene therapy contexts. A large variety of such vectors are known in the art and are generally available.

In some cases, a nucleic acid encoding resistance to kanamycin may be include but is not limited to sequences such as:

(SEQ ID NO: 5)
ttcgcatgat tgaacaagat ggattgcacg caggttctcc
ggccgcttgg gtggagaggc tattcggcta tgactgggca
caacagacaa tcggctgctc tgatgccgcc gtgttccggc
tgtcagcgca ggggcgcccg gttctttttg tcaagaccga
cctgtccggt gccctgaatg aactgcaaga cgaggcagcg
cggctatcgt ggctggccac gacgggcgtt ccttgcgcag
ctgtgctcga cgttgtcact gaagcgggaa gggactggct
gctattgggc gaagtgccgg ggcaggatct cctgtcatct
caccttgctc ctgccgagaa agtatccatc atggctgatg
caatgcggcg gctgcatacg cttgatccgg ctacctgccc
attcgaccac caagcgaaac atcgcatcga gcgagcacgt
actcggatgg aagccggtct tgtcgatcag gatgatctgg
acgaagagca tcaggggctc gcgccagccg aactgttcgc
caggctcaag gcgagcatgc ccgacggcga ggatctcgtc
gtgacccatg gcgatgcctg cttgccgaat atcatggtgg
aaaatggccg cttttctgga ttcatcgact gtggccggct
gggtgtggcg gaccgctatc aggacatagc gttggctacc
cgtgatattg ctgaagagct tggcggcgaa tgggctgacc
gcttcctcgt gctttacggt atcgccgctc ccgattcgca
gcgcatcgcc ttctatcgcc ttcttgacga gttcttctga.

The recombinant vector/plasmid comprise a polynucleotide encoding a human Maxi-K protein, a mutant Maxi-K protein or a functional fragment thereof. An Exemplary nucleic acid encoding the Maxi-K protein suitable for use in the present invention include the nucleic acid sequence of SEQ ID NO: 6.

hSlo
(SEQ ID NO: 6)
ATGGCAAACGGTGGCGGCGGCGGCGGCGGCAGCAGCGGCGGCGGCGGCGGC
GGCGGCGGAGGCAGCGGTCTTAGAATGAGCAGCAATATCCACGCGAACCAT
CTCAGCCTAGACGCGTCCTCCTCCTCCTCCTCCTCCTCTTCCTCTTCTTCT
TCTTCCTCCTCCTCTTCCTCCTCGTCCTCGGTCCACGAGCCCAAGATGGAT
GCGCTCATCATCCCGGTGACCATGGAGGTGCCGTGCGACAGCCGGGGCCAA
CGCATGTGGTGGGCTTTCCTGGCCTCCTCCATGGTGACTTTCTTCGGGGGC
CTCTTCATCATCTTGCTCIGGCGGACGCTCAAGTACCTGIGGACCGTGTGC
TGCCACTGCGGGGGCAAGACGAAGGAGGCCCAGAAGATTAACAATGGCTCA
AGCCAGGCGGATGGCACTCTCAAACCAGTGGATGAAAAAGAGGAGGCAGTG
GCCGCCGAGGTCGGCTGGATGACCTCCGTGAAGGACTGGGCGGGGGTGATG
ATATCCGCCCAGACACTGACTGGCAGAGTCCTGGTTGTCTTAGTCTTTGCT
CTCAGCATCGGTGCACTTGTAATATACTTCATAGATTCATCAAACCCAATA
GAATCCTGCCAGAATTTCTACAAAGATTTCACATTACAGATCGACATGGCT
TTCAACGTGTTCTTCCTTCTCTACTTTGGCTTGCGGTTTATTGCAGCCAAC
GATAAATTGTGGTTCTGGCTGGAAGTGAACTCTGTAGTGGATTTCTTCACG
GTGCCCCCCGTGTTTGTGTCTGTGTACTTAAACAGAAGTTGGCTTGGTTTG
AGATTTTTAAGAGCTCTGAGACTGATACAGTTTTCAGAAATTTTGCAGTTT
CTGAATATTCTTAAAACAAGTAATTCCATCAAGCTGGTGAATCTGCTCTCC
ATATTTATCAGCACGTGGCTGACTGCAGCTGGGTTCATCCATTTGGTGGAG
AATTCAGGGGACCCATGGGAAAATTTCCAAAACAACCAGGCTCTCACCTAC
TGGGAATGTGTCATTTACTCATGGTCACAATGTCCACCGTTGGTTATGGGG
ATGTTTATGCAAAAACCACACTTCGGCGCCTCTTCATGGTCTTCTTCATCC
TCGGGGGACTGGCCATGTTTGCCAGCTACGTCCCTGAAATCATAGAGTTAA
TAGGAAACCGCAAGAAATACGGGGGCTCCTATAGTGCGGTTAGTGGAAGAA
AGCACATTGTGGTCTGCGGACACATCACTCTGGAGAGTGTTTCCAACTTCC
TGAAGGACTTTCTGCACAAGGACCGGGATGACGTCAATGTGGAGATCGTTT
TTCTTCACAACATCTCCCCCAACCTGGAGCTTGAAGCTCTGTTCAAACGAC
ATTTTACTCAGGTGGAATTTTATCAGGGTTCCGTCCTCAATCCACATGATC
TTGCAAGAGTCAAGATAGAGTCAGCAGATGCATGCCTGATCCTTGCCAACA
AGTACTGCGCTGACCCGGATGCGGAGGATGCCTCGAATATCATGAGAGTAA
TCTCCATAAAGAACTACCATCCGAAGATAAGAATCATCACTCAAATGCTGC
AGTATCACAACAAGGCCCATCTGCTAAACATCCGAGCTGGAATTGGAAAGA
AGGTGATGACGCAATCTGCCTCGCAGAGTTGAAGTTGGGCTTCATAGCCCA
GAGCTGCCTGGCTCAAGGCCTCTCCACCATGCTTGCCAACCTTCTCCATGA
GGTCATTCATAAAGATTGAGGAAGACACATGGCAGAAATACTACTTGGAAG
GAGTCTCAAATCAAATGTACACAGAATATCTCTCCAGTGCCTTCGTGGGTC
TGTCCTTCCCTACTGTTTGTGAGCTGTGTTTTGTGAAGCTCAAGCTCCTAA
TGATAGCCATTGAGTACAAGTCTGCCAACCGAGAGAGCCGTATATTAATTA
ATCCTGGAAACCATTTTAAGATCCAAGAAGGTACTTTAGGATTTTTCATCG
CAAGTGATGCCAAAGAAGTTAAAAGGGCATTTTTTTACTGCAAGGCCTGTC
ATGATGACATCACAGATCCCAAAAGAATAAAAAAATGTGGCTGCAAACGGC
TTGAAGATGAGCAGCCGTCAACACTATCACCAAAAAAAAAGCAACGGAATG
GAGGCATGCGGAACTCACCCAACACCTCGCCTAAGCTGATGAGGCATGACC
CCTTGTTAATTCCTGGCAATGATCAGATTGACAACATGGACTCCAATGTGA
AGAAGTACGACTCTACTGGGATGTTTCACTGGTGTGCACCCAAGGAGATAG
AGAAAGTCATCCTGACTCGAAGTGAAGCTGCCATGACCGTCCTGAGTGGCC
ATGTCGTGGTCTGCATCTTTGGCGACGTCAGCTCAGCCCTGATCGGCCTCC
GGAACCTGGTGATGCCGCTCCGTGCCAGCAACTTTCATTACCATGAGCTCA
AGCACATTGTGTTTGTGGGCTCTATTGAGTACCTCAAGCGGGAATGGGAGA
CGCTTCATAACTTCCCCAAAGTGTCCATATTGCCTGGTACGCCATTAAGTC
GGGCTGATTTAAGGGCTGTCAACATCAACCTCTGTGACATGTGCGTTATCC
TGTCAGCCAATCAGAATAATATTGATGATACTTCGCTGCAGGACAAGGAAT
GCATCTTGGCGTCACTCAACATCAAATCTATGCAGTTTGATGACAGCATCG
GAGTCTTGCAGGCTAATTCCCAAGGGTTCACACCTCCAGGAATGGATAGAT
CCTCTCCAGATAACAGCCCAGTGCACGGGATGTTACGTCAACCATCCATCA
CAACTGGGGTCAACATCCCCATCATCACTGAACTAGTGAACGATACTAATG
TTCAGTTTTTGGACCAAGACGATGATGATGACCCTGATACAGAACTGTACC
TCACGCAGCCCTTTGCCTGTGGGACAGCATTTGCCGTCAGTGTCCTGGACT
CACTCATGAGCGCGACGTACTTCAATGACAATATCCTCACCCTGATACGGA
CCCTGGTGACCGGAGGAGCCACGCCGGAGCTGGAGGCTCTGATTGCTGAGG
AAAACGCCCTTAGAGGTGGCTACAGCACCCCGCAGACACTGGCCAATAGGG
ACCGCTGCCGCGTGGCCCAGTTAGCTCTGCTCGATGGGCCATTTGCGGACT
TAGGGGATGGTGGTTGTTATGGTGATCTGTTCTGCAAAGCTCTGAAAACAT
ATAATATGCTTTGTTTTGGAATTTACCGGCTGAGAGATGCTCACCTCAGCA
CCCCCAGTCAGTGCACAAAGAGGTATGTCATCACCAACCCGCCCTATGAGT
TTGAGCTCGTGCCGACGGACCTGATCTTCTGCTTAATGCAGTTTGACCACA
ATGCCGGCCAGTCCCGGGCCAGCCTGTCCCATTCCTCCCACTCGTCGCAGT
CCTCCAGCAAGAAGAGCTCCTCTGTTCACTCCATCCCATCCACAGCAAACC
GACAGAACCGGCCCAAGTCCAGGGAGTCCCGGGACAAACAGAAGTACGTGC
AGGAAGAGCGGCTTTGATATGTGTATCCACCGCCACTGTGTGAAACTGTAT
CTGCCACTCATTTCCCCAGTTGGTGTTTCCAACAAAGTAACTTTCCCTGTT
TTCCCCTGTAGTCCCCCCCTTTTTTTTTACACATATTTGCATATGTATGAT
AGTGTGCATGTGGTTGTCATTTTTATTTCACCACCATAAAACCCTTGAGCA
CAACAGCAAATAAGCAGACGGGCTCCGGAATTCCTGCAGCCCGGGGGATCC
ACTAG

Modifications of the hSlo gene may be used to effectively treat human disease that is caused, for example, by alterations of the BK channel expression, activity, upstream signaling events, and/or downstream signaling events. Modifications to a wild type nucleotide or peptide sequence of hSlo may include, but are not limited to, deletions, insertions, frameshifts, substitutions, and inversions. For example, contemplated modifications to the wild type sequence of hSlo include substitutions of a single nucleotide in a DNA, cDNA, or RNA sequence encoding hSlo and/or substitutions of a single amino acid in a peptide or polypeptide sequence encoding hSlo. The substitution of a single nucleotide in a DNA, cDNA, or RNA sequence encoding hSlo and/or a single amino acid in a peptide or polypeptide sequence encoding hSlo is also referred to as a point mutation. Substitutions within a DNA, cDNA, or RNA sequence encoding hSlo and/or a peptide or polypeptide sequence encoding hSlo may be conserved or non-conserved.

Preferred modification in the hSlo gene include a point mutation at nucleic acid position 1054 when numbered in accordance with SEQ ID NO: 7. This point mutation results in an amino acid substitution at position 352 of the MaxiK Channel protein when numbered in accordance with SEQ ID NO: 7. For example the point mutation is a substitution of a Serine (S) for a Threonine (T) (e.g., T352S). Optionally, additional modifications in the hSlo gene include point mutations that result in one or more amino acid substitution at amino acid positions 496, 602, 681, 778, 805 or 977 when numbered in accordance with SEQ ID NO: 8.

Additional mutations in the amino acid sequence (SEQ ID NO: 8) are also highlighted by white lettering on a black background and accompanied by the name of the mutation (e.g. C977A (C1), C496A (C2), C681A (C3), M602L (M1), M778L (M2) and M805L (M3)).

| 1 | ATGGCAAATGGTGGCGGCGGCGGCGGCGGCAGCAGCGGCGGCGGCGGCGGCGGCGGAGGC | 60 |
|---|---|---|
| 1 | M A N G G G G G G G S S G G G G G G G G | |
| 61 | AGCAGTCTTAGAATGAGTAGCAATATCCACGCGAACCATCTCAGCCTAGACGTGTCCTCC | 120 |
| 21 | S S L R M S S N I H A N H L S L D V S S | |
| 121 | TCCTCCTCCTCCTCCTCTTCCTCTTCTTCTTCTTCCTCCTCCTCTTCCTCCTCGTCCTCG | 180 |
| 41 | S S S S S S S S S S S S S S S S S S S S | |
| 181 | GTCCACGAGCCCAAGATGGATGCGCTCATCATCCCGGTGACCATGGAGGTGCCGTGCGAC | 240 |
| 61 | V H E P K M D A L I I P V T M E V P C D | |
| 241 | AGCCGGGGCCAACGCATGTGGTGGGCTTTCCTGGCCTCCTCCATGGTGACTTTCTTCGGG | 300 |
| 81 | S R G Q R M W W A F L A S S M V T F F G | |
| 301 | GGCCTCTTCATCATCTTGCTCTGGCGGACGCTCAAGTACCTGTGGACCGTGTGCTGCCAC | 360 |
| 101 | G L F I I L L W R T L K Y L W T V C C H | |
| 361 | TGCGGGGGCAAGACGAAGGAGGCCCAGAAGATTAACAATGGCTCAAGCCAGGCGGATGGC | 420 |
| 121 | C G G K T K E A Q K I N N G S S Q A D G | |
| 421 | ACTCTCAAACCAGTGGATGAAAAAGAGGAGGCAGTGGCCGCCGAGGTCGGCTGGATGACC | 480 |
| 141 | T L K P V D E K E E A V A A E V G W M T | |
| 481 | TCCGTGAAGGACTGGGCGGGGGTGATGATATCCGCCCAGACACTGACTGGCAGAGTCCTG | 540 |
| 161 | S V K D W A G V M I S A Q T L T G R V L | |
| 541 | GTTGTCTTAGTCTTTGCTCTCAGCATCGGTGCACTTGTAATATACTTCATAGATTCATCA | 600 |
| 181 | V V L V F A L S I G A L V I Y F I D S S | |
| 601 | AACCCAATAGAATCCTGCCAGAATTTCTACAAAGATTTCACATTACAGATCGACATGGCT | 660 |
| 201 | N P I E S C Q N F Y K D F T L Q I D M A | |
| 661 | TTCAACGTGTTCTTCCTTCTCTACTTCGGCTTGCGGTTTATTGCAGCCAACGATAAATTG | 720 |
| 221 | F N V F F L L Y F G L R F I A A N D K L | |
| 721 | TGGTTCTGGCTGGAAGTGAACTCTGTAGTGGATTTCTTCACGGTGCCCCCCGTGTTTGTG | 780 |
| 241 | W F W L E V N S V V D F F T V P P V F V | |
| 781 | TCTGTGTACTTAAACAGAAGTTGGCTTGGTTTGAGATTTTTAAGAGCTCTGAGACTGATA | 840 |
| 261 | S V Y L N R S W L G L R F L R A L R L I | |
| 841 | CAGTTTTCAGAAATTTTGCAGTTTCTGAATATTCTTAAAACAAGTAATTCCATCAAGCTG | 900 |
| 281 | Q F S E I L Q F L N I L K T S N S I K L | |
| 901 | GTGAATCTGCTCTCCATATTTATCAGCACGTGGCTGACTGCAGCCGGGTTCATCCATTTG | 960 |
| 301 | V N L L S I F I S T W L T A A G F I H L | |
| 961 | GTGGAGAATTCAGGGGACCCATGGGAAAATTTCCAAAACAACCAGGCTCTCACCTACTGG | 1020 |
| 321 | V E N S G D P W E N F Q N N Q A L T Y W | |
| 1021 | GAATGTGTCTATTTACTCATGGTCACAATGTCCACCGTTGGTTATGGGGATGTTTATGCA | 1080 |
| | 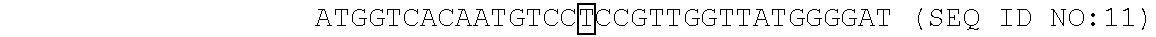 | |
| 341 | 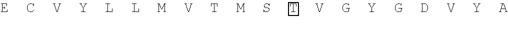 | |
| 1081 | AAAACCACACTTGGGCGCCTCTTCATGGTCTTCTTCATCCTCGGGGGACTGGCCATGTTT | 1140 |
| 361 | K T T L G R L F M V F F I L G G L A M F | |
| 1141 | GCCAGCTACGTCCCTGAAATCATAGAGTTAATAGGAAACCGCAAGAAATACGGGGGCTCC | 1200 |
| 381 | A S Y V P E I I E L I G N R K K Y G G S | |
| 1201 | TATAGTGCGGTTAGTGGAAGAAAGCACATTGTGGTCTGCGGACACATCACTCTGGAGAGT | 1260 |
| 401 | Y S A V S G R K H I V V C G H I T L E S | |
| 1261 | GTTTCCAACTTCCTGAAGGACTTTCTGCACAAGGACCGGGATGACGTCAATGTGGAGATC | 1320 |
| 421 | V S N F L K D F L H K D R D D V N V E I | |
| 1321 | GTTTTTCTTCACAACATCTCCCCCAACCTGGAGCTTGAAGCTCTGTTCAAACGACATTTT | 1380 |
| 441 | V F L H N I S P N L E L E A L F K R H F | |
| 1381 | ACTCAGGTGGAATTTTATCAGGGTTCCGTCCTCAATCCACATGATCTTGCAAGAGTCAAG | 1440 |
| 461 | T Q V E F Y Q G S V L N P H D L A R V K | |
| 1441 | ATAGAGTCAGCAGATGCATGCCTGATCCTTGCCAACAAGTACTGCGCTGACCCGGATGCG | 1500 |
| 481 | 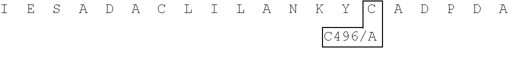 | |
| 1501 | GAGGATGCCTCGAATATCATGAGAGTAATCTCCATAAAGAACTACCATCCGAAGATAAGA | 1560 |
| 501 | E D A S N I M R V I S I K N Y H P K I R | |
| 1561 | ATCATCACTCAAATGCTGCAGTATCACAACAAGGCCCATCTGCTAAACATCCCGAGCTGG | 1620 |
| 521 | I I T Q M L Q Y H N K A H L L N I P S W | |
| 1621 | AATTGGAAAGAAGGTGATGACGCAATCTGCCTCGCAGAGTTGAAGTTGGGCTTCATAGCC | 1680 |
| 541 | N W K E G D D A I C L A E L K L G F I A | |
| 1681 | CAGAGCTGCCTGGCTCAAGGCCTCTCCACCATGCTTGCCAACCTCTTCTCCATGAGGTCA | 1740 |
| 561 | Q S C L A Q G L S T M L A N L F S M R S | |
| 1741 | TTCATAAAGATTGAGGAAGACACATGGCAGAAATACTACTTGGAAGGAGTCTCAAATGAA | 1800 |
| 581 | F I K I E E D T W Q K Y Y L E G V S N E | |
| 1801 | ATGTACACAGAATATCTCTCCAGTGCCTTCGTGGGTCTGTCCTTCCCTACTGTTTGTGAG | 1860 |
| 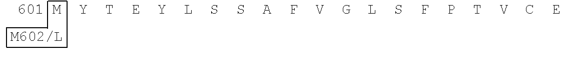 | | |
| 1861 | CTGTGTTTTGTGAAGCTCAAGCTCCTAATGATAGCCATTGAGTACAAGTCTGCCAACCGA | 1920 |
| 621 | L C F V K L K L L M I A I E Y K S A N R | |
| 1921 | GAGAGCCGTATATTAATTAATCCTGGAAACCATCTTAAGATCCAAGAAGGTACTTTAGGA | 1980 |
| 641 | E S R I L I N P G N H L K I Q E G T L G | |
| 1981 | TTTTTCATCGCAAGTGATGCCAAAGAAGTTAAAAGGGCATTTTTTTACTGCAAGGCCTGT | 2040 |
| 661 | 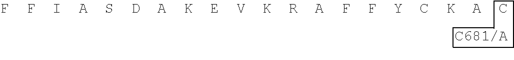 | |
| 2041 | CATGATGACATCACAGATCCCAAAAGAATAAAAAAATGTGGCTGCAAACGGCTTGAAGAT | 2100 |
| 681 | H D D I T D P K R I K K C G C K R L E D | |
| 2101 | GAGCAGCCGTCAACACTATCACCAAAAAAAAAGCAACGGAATGGAGGCATGCGGAACTCA | 2160 |
| 701 | E Q P S T L S P K K K Q R N G G M R N S | |
| 2161 | CCCAACACCTCGCCTAAGCTGATGAGGCATGACCCCTTGTTAATTCCTGGCAATGATCAG | 2220 |
| 721 | P N T S P K L M R H D P L L I P G N D Q | |
| 2221 | ATTGACAACATGGACTCCAATGTGAAGAAGTACGACTCTACTGGGATGTTTCACTGGTGT | 2280 |
| 741 | I D N M D S N V K K Y D S T G M F H W C | |
| 2281 | GCACCCAAGGAGATAGAGAAAGTCATCCTGACTCGAAGTGAAGCTGCCATGACCGTCCTG | 2340 |
| 761 | 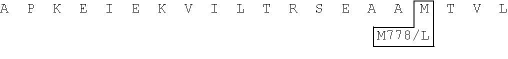 | |
| 2341 | AGTGGCCATGTCGTGGTCTGCATCTTTGGCGACGTCAGCTCAGCCCTGATCGGCCTCCGG | 2400 |
| 781 | S G H V V V C I F G D V S S A L I G L R | |
| 2401 | AACCTGGTGATGCCGCTCCGTGCCAGCAACTTTCATTACCATGAGCTCAAGCACATTGTG | 2460 |
| 801 | 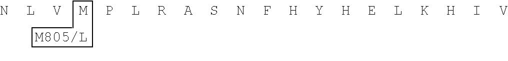 | |
| 2461 | TTTGTGGGCTCTATTGAGTACCTCAAGCGGGAATGGGAGACGCTTCATAACTTCCCCAAA | 2520 |
| 821 | F V G S I E Y L K R E W E T L H N F P K | |
| 2521 | GTGTCCATATTGCCTGGTACGCCATTAAGTCGGGCTGATTTAAGGGCTGTCAACATCAAC | 2580 |
| 841 | V S I L P G T P L S R A D L R A V N I N | |
| 2581 | CTCTGTGACATGTGCGTTATCCTGTCAGCCAATCAGAATAATATTGATGATACTTCGCTG | 2640 |
| 861 | L C D M C V I L S A N Q N N I D D T S L | |
| 2641 | CAGGACAAGGAATGCATCTTGGCGTCACTCAACATCAAATCTATGCAGTTTGATGACAGC | 2700 |
| 881 | Q D K E C I L A S L N I K S M Q F D D S | |
| 2701 | ATCGGAGTCTTGCAGGCTAATTCCCAAGGGTTCACACCTCCAGGAATGGATAGATCCTCT | 2760 |
| 901 | I G V L Q A N S Q G F T P P G M D R S S | |
| 2761 | CCAGATAACAGCCCAGTGCACGGGATGTTACGTCAACCATCCATCACAACTGGGGTCAAC | 2820 |
| 921 | P D N S P V H G M L R Q P S I T T G V N | |
| 2821 | ATCCCCATCATCACTGAACTAGTGAACGATACTAATGTTCAGTTTTTGGACCAAGACGAT | 2880 |
| 941 | I P I I T E L V N D T N V Q F L D Q D D | |
| 2881 | GATGATGACCCTGATACAGAACTGTACCTCACGCAGCCCTTTGCCTGTGGGACAGCATTT | 2940 |
| 961 | 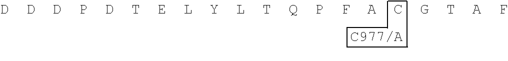 | |
| 2941 | GCCGTCAGTGTCCTGGACTCACTCATGAGCGCGACGTACTTCAATGACAATATCCTCACC | 3000 |
| 981 | A V S V L D S L M S A T Y F N D N I L T | |
| 3001 | CTGATACGGACCCTGGTGACCGGAGGAGCCACGCCGGAGCTGGAGGCTCTGATTGCTGAG | 3060 |
| 1001 | L I R T L V T G G A T P E L E A L I A E | |
| 3061 | GAAAACGCCCTTAGAGGTGGCTACAGCACCCCGCAGACACTGGCCAATAGGGACCGCTGC | 3120 |
| 1021 | E N A L R G G Y S T P Q T L A N R D R C | |
| 3121 | CGCGTGGCCCAGTTAGCTCTGCTCGATGGGCCATTTGCGGACTTAGGGGATGGTGGTTGT | 3180 |
| 1041 | R V A Q L A L L D G P F A D L G D G G C | |
| 3181 | TATGGTGATCTGTTCTGCAAAGCTCTGAAAACATATAATATGCTTTGTTTTGGAATTTAC | 3240 |
| 1061 | Y G D L F C K A L K T Y N M L C F G I Y | |
| 3241 | CGGCTGAGAGATGCTCACCTCAGCACCCCCAGTCAGTGCACAAAGAGGTATGTCATCACC | 3300 |
| 1081 | R L R D A H L S T P S Q C T K R Y V I T | |
| 3301 | AACCCGCCCTATGAGTTTGAGCTCGTGCCGACGGACCTGATCTTCTGCTTAATGCAGTTT | 3360 |
| 1101 | N P P Y E F E L V P T D L I F C L M Q F | |
| 3361 | GACCACAATGCCGGCCAGTCCCGGGCCAGCCTGTCCCATTCCTCCCACTCGTCGCAGTCC | 3420 |
| 1121 | D H N A G Q S R A S L S H S S H S S Q S | |
| 3421 | TCCAGCAAGAAGAGCTCCTCTGTTCACTCCATCCCATCCACAGCAAACCGACAGAACCGG | 3480 |
| 1141 | S S K K S S S V H S I P S T A N R Q N R | |
| 3481 | CCCAAGTCCAGGGAGTCCCGGGACAAACAGAAGTACGTGCAGGAAGAGCGGCTT (SEQ. ID NO: 7) | 3538 |
| 1161 | P K S R E S R D K Q K Y V Q E E R L (SEQ ID NO: 8) | |

The present invention further provides a smooth muscle cell which expresses an exogenous DNA sequence encoding a protein involved in the regulation of smooth muscle tone. As used herein, “exogenous” means any DNA that is introduced into an organism or cell. In some embodiments, the exogenous DNA sequence encodes hSlo.

Pharmaceutical Compositions

A pharmaceutical composition is a formulation containing one or more active ingredients as well as one or more excipients, carriers, stabilizers or bulking agents, which is suitable for administration to a human patient to achieve a desired diagnostic result or therapeutic or prophylactic effect. For storage stability and convenience of handling, a pharmaceutical composition can be formulated as a lyophilized (i.e. freeze dried) or vacuum dried powder which can be reconstituted with saline or water prior to administration to a patient. Alternately, the pharmaceutical composition can be formulated as an aqueous solution. A pharmaceutical composition can contain a proteinaceous active ingredient. Various excipients, such as albumin and gelatin have been used with differing degrees of success to try and stabilize a protein active ingredient present in a pharmaceutical composition. Additionally, cryoprotectants such as alcohols have been used to reduce protein denaturation under the freezing conditions of lyophilization.

Pharmaceutical compositions suitable for internal use include sterile aqueous solutions or dispersions and sterile powders for the extemporaneous preparation of sterile injectable solutions or dispersion. For intravenous administration, suitable carriers include physiological saline, bacteriostatic water, or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants such as polysorbates (Tween.™.), sodium dodecyl sulfate (sodium lauryl sulfate), lauryl dimethyl amine oxide, cetyltrimethylammonium bromide (CTAB), polyethoxylated alcohols, polyoxyethylene sorbitan, octoxynol (Triton X100.™.), N,N-dimethyldodecylamine-N-oxide, hexadecyltrimethylammonium bromide (HTAB), polyoxyl 10 lauryl ether, Brij 721.™., bile salts (sodium deoxycholate, sodium cholate), pluronic acids (F-68, F-127), polyoxyl castor oil (Cremophor.™.) nonylphenol ethoxylate (Tergitol.™.), cyclodextrins and, ethylbenzethonium chloride (Hyamine.™.) Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the internal compositions can be brought about by including in the composition an agent which delays absorption, for example, aluminum monostearate and gelatin.

Sterile solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

The pharmaceutical compositions can be included in a container, pack, or dispenser together with instructions for administration.

Certain compositions of the present disclosure also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The co-administration of a nucleic acid and a carrier compound, generally with an excess of the latter substance, can result in a substantial reduction of the amount of nucleic acid recovered in the liver, kidney or other extra circulatory reservoirs, presumably due to competition between the carrier compound and the nucleic acid for a common receptor. For example, the recovery of a partially phosphorothioate oligonucleotide in hepatic tissue can be reduced when it is co-administered with polyinosinic acid, dextran sulphate, polycytidic acid or 4-acetamido-4′isothiocyano-stilbene-2,2′disulfonic acid (Miyao et al., Antisense Res. Dev., 1995, 5, 115-121; Takakura et al., Antisense & Nucl. Acid Drug Dev., 1996, 6, 177-183).

The vector can be incorporated into pharmaceutical compositions for administration to mammalian patients, particularly humans. The vector or virions can be formulated in nontoxic, inert, pharmaceutically acceptable aqueous carriers, preferably at a pH ranging from 3 to 8, more preferably ranging from 6 to 8, most preferably ranging from 6.8 to 7.2. Such sterile compositions will comprise the vector containing the nucleic acid encoding the therapeutic molecule dissolved in an aqueous buffer having an acceptable pH upon reconstitution.

In some aspects, the pharmaceutical compositions provided herein comprise a therapeutically effective amount of a vector in admixture with a pharmaceutically acceptable carrier and/or excipient, for example saline, phosphate buffered saline, phosphate and amino acids, polymers, polyols, sugar, buffers, preservatives and other proteins. Exemplary amino acids, polymers and sugars and the like are octylphenoxy polyethoxy ethanol compounds, polyethylene glycol monostearate compounds, polyoxyethylene sorbitan fatty acid esters, sucrose, fructose, dextrose, maltose, glucose, mannitol, dextran, sorbitol, inositol, galactitol, xylitol, lactose, trehalose, bovine or human serum albumin, citrate, acetate, Ringer's and Hank's solutions, cysteine, arginine, carnitine, alanine, glycine, lysine, valine, leucine, polyvinylpyrrolidone, polyethylene and glycol.

In some aspects, the pharmaceutical composition provided herein comprises a buffer, such as phosphate buffered saline (PBS) or sodium phosphate/sodium sulfate, tris buffer, glycine buffer, sterile water and other buffers known to the ordinarily skilled artisan such as those described by Good et al. (1966) Biochemistry 5:467. Preferred pharmaceutical composition contains sodium phosphate, sodium chloride and sucrose.

In some aspects, the pharmaceutical composition provided herein comprises substances which increase the viscosity of the suspension, such as sodium carboxymethyl cellulose, sorbitol, sucrose or dextran, in the amount about 1-36 percent, such as 1, 2, 3, 4, 5, 6, 7, 8, 9, 10, 11, 12, 13, 14, 15, 16, 17, 18, 19 or 26 percent (v/v). Preferably the sucrose is about 10-30% (v/v), most preferably the sucrose is about 20%. (v/v).

Prior to administration the pharmaceutical composition is free of components used during the production, e.g., culture components, host cell protein, host cell DNA, plasmid DNA and substantially free of mycoplasm, endotoxin, and microbial contamination. Preferably, the pharmaceutical composition has less than 10, 5, 3, 2, or 1 CFU/swab. Most preferably composition has 0 CFU/swab. The endotoxin level in the pharmaceutical composition is less than 26 EU/mL, less than 10 EU/mL or less than 5 EU/mL.

Kits

Compositions and reagents useful for the present disclosure may be packaged in kits to facilitate application of the present disclosure. In some aspects, the present method provides for a kit comprising a recombinant nucleic acid of the disclosure. In some aspects, the present method provides for a kit comprising a recombinant virus of the disclosure. The instructions could be in any desired form, including but not limited to, printed on a kit insert, printed on one or more containers, as well as electronically stored instructions provided on an electronic storage medium, such as a computer readable storage medium. Also optionally included is a software package on a computer readable storage medium that permits the user to integrate the information and calculate a control dose.

In another aspect, the present disclosure provides a kit comprising the pharmaceutical compositions provided herein. In yet another aspect, the disclosure provides kits in the treatment of diseases.

In one aspect, a kit comprises: (a) a recombinant virus provided herein, and (b) instructions to administer to cells or an individual a therapeutically effective amount of the recombinant virus. In some aspects, the kit may comprise pharmaceutically acceptable salts or solutions for administering the recombinant virus. Optionally, the kit can further comprise instructions for suitable operational parameters in the form of a label or a separate insert. For example, the kit may have standard instructions informing a physician or laboratory technician to prepare a dose of recombinant virus.

Optionally, the kit may further comprise a standard or control information so that a patient sample can be compared with the control information standard to determine if the test amount of recombinant virus is a therapeutic amount Optionally, the kit could further comprise devices for administration, such as a syringe, filter needle, extension tubing, and cannula.

### Definitions

The compositions and methods of this disclosure as described herein may employ, unless otherwise indicated, conventional techniques and descriptions of molecular biology (including recombinant techniques), cell biology, biochemistry, immunochemistry and ophthalmic techniques, which are within the skill of those who practice in the art. Such conventional techniques include methods for observing and analyzing the retina, or vision in a subject, cloning and propagation of recombinant virus, formulation of a pharmaceutical composition, and biochemical purification and immunochemistry. Specific illustrations of suitable techniques can be had by reference to the examples herein. However, equivalent conventional procedures can, of course, also be used. Such conventional techniques and descriptions can be found in standard laboratory manuals such as Green, et al., Eds., Genome Analysis: A Laboratory Manual Series (Vols. I-IV) (1999); Weiner, et al., Eds., Genetic Variation: A Laboratory Manual (2007); Dieffenbach, Dveksler, Eds., PCR Primer: A Laboratory Manual (2003); Bowtell and Sambrook, DNA Microarrays: A Molecular Cloning Manual (2003); Mount, Bioinformatics: Sequence and Genome Analysis (2004); Sambrook and Russell, Condensed Protocols from Molecular Cloning: A Laboratory Manual (2006); and Sambrook and Russell, Molecular Cloning: A Laboratory Manual (2002) (all from Cold Spring Harbor Laboratory Press); Stryer, L., Biochemistry (4th Ed.) W.H. Freeman, N.Y. (1995); Gait, “Oligonucleotide Synthesis: A Practical Approach” IRL Press, London (1984); Nelson and Cox, Lehninger, Principles of Biochemistry, 3rd Ed., W.H. Freeman Pub., New York (2000); and Berg et al., Biochemistry, 5th Ed., W.H. Freeman Pub., New York (2002), all of which are herein incorporated by reference in their entirety for all purposes.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

Ranges can be expressed herein as from “about” one particular value, and/or to “about” another particular value. When such a range is expressed, another case includes from the one particular value and/or to the other particular value. Similarly, when values are expressed as approximations, by use of the antecedent “about,” it will be understood that the particular value forms another case. It will be further understood that the endpoints of each of the ranges are significant both in relation to the other endpoint, and independently of the other endpoint. The term “about” as used herein refers to a range that is 15% plus or minus from a stated numerical value within the context of the particular usage. For example, about 10 would include a range from 8.5 to 11.5. The term “about” also accounts for typical error or imprecision in measurement of values.

In the context of the invention, the term “treating” or “treatment”, as used herein, means reversing, alleviating, inhibiting the progress of, or preventing the disorder or condition to which such term applies, or one or more symptoms of such disorder or condition (e.g., idiopathic overactive bladder syndrome).

According to the invention, the term “patient” or “patient in need thereof”, is intended for a human or non-human mammal affected or likely to be affected with idiopathic overactive bladder syndrome.

As used herein, the term “detrusor” or “detrusor muscle” is meant the muscle of the bladder. By “intradetrusorally” is meant into the detrusor muscle.

As intended herein the expression “isolated nucleic acid” refers to any type of isolated nucleic acid, it can notably be natural or synthetic, DNA or RNA, single or double stranded. In particular, where the nucleic acid is synthetic, it can comprise non-natural modifications of the bases or bonds, in particular for increasing the resistance to degradation of the nucleic acid. Where the nucleic acid is RNA, the modifications notably encompass capping its ends or modifying the 2′ position of the ribose backbone so as to decrease the reactivity of the hydroxyl moiety, for instance by suppressing the hydroxyl moiety (to yield a 2′-deoxyribose or a 2′-deoxyribose-2′-fluororibose), or substituting the hydroxyl moiety with an alkyl group, such as a methyl group (to yield a 2′-O-methyl-ribose).

Two amino acid sequences or nucleic acid sequences are “substantially homologous” or “substantially similar” when greater than 80%0, preferably greater than 85%, preferably greater than 90% of the amino acids or nucleic acid sequences are identical, or greater than about 900%, preferably greater than 95%0, are similar (functionally identical). To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences. In one embodiment, the two sequences are the same length. The determination of percent identity between two sequences can be accomplished using a mathematical algorithm. Preferably, the similar or homologous sequences are identified by alignment using, for example, the GCG (Genetics Computer Group, Program Manual for the GCG Package, Version 7, Madison, Wis.) pileup program, or any of sequence comparison algorithms such as BLAST, FASTA, etc.

As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

### EXAMPLES

### Example 1: Non-Clinical Studies with of HMAXI-K Gene Transfer

### Rats

The pathophysiology of partial urinary outlet obstruction in the rat model recapitulates many relevant aspects of the corresponding lower urinary tract symptoms observed in humans. The noted physiological and pathophysiological similarities make it reasonable to assume that studies on the rat bladder will provide insight into at least some aspects of human bladder physiology and dysfunction.

Because the physiology of the rat bladder parallels many aspects of the human bladder studies examined the potential utility of bladder instilled K channel gene therapy with hSlo cDNA (i.e., the maxi-K channel) to ameliorate bladder overactivity in a rat model of partial urinary outlet obstruction.

In one study, twenty-two female Sprague-Dawley rats were subjected to partial urethral (i.e., outlet, PUO) obstruction, with 17 sham-operated control rats run in parallel. After 6 weeks of obstruction, suprapubic catheters were surgically placed in the dome of the bladder in all rats. Twelve obstructed rats received bladder instillation of 100 μg of hSlo/pcDNA in 1 ml PBS-20% sucrose during catheterization and another 10 obstructed rats received 1 ml PBS-20% sucrose (7 rats) or 1 ml PBS-20% sucrose containing pcDNA only (3 rats). Two days after surgery cystometry was performed on all animals to examine the characteristics of the micturition reflex in conscious and unrestrained rats. Obstruction was associated with a three to fourfold increase in bladder weight and alterations in virtually every micturition parameter estimate (see Table 1).

Obstructed rats injected with PBS-20% sucrose routinely displayed spontaneous bladder contractions between micturitions. In contrast, hSlo injection eliminated the obstruction-associated bladder hyperactivity, without detectably affecting any other cystometric parameter. Presumably, expression of hSlo in rat bladder functionally antagonizes the increased contractility normally observed in obstructed animals and thereby ameliorates bladder overactivity.

Another study examined the ability of hSlo gene transfer to alter and/or ameliorate the intermicturition pressure fluctuations observed in an obstructed male rat model. For these studies rats were obstructed for 2 weeks using a perineal approach. Following 2 weeks of obstruction, the rats were catheterized for cystometric investigations and placed into 1 of 2 treatment groups. Age-Matched Control rats were subjected to a sham obstruction and run in parallel.

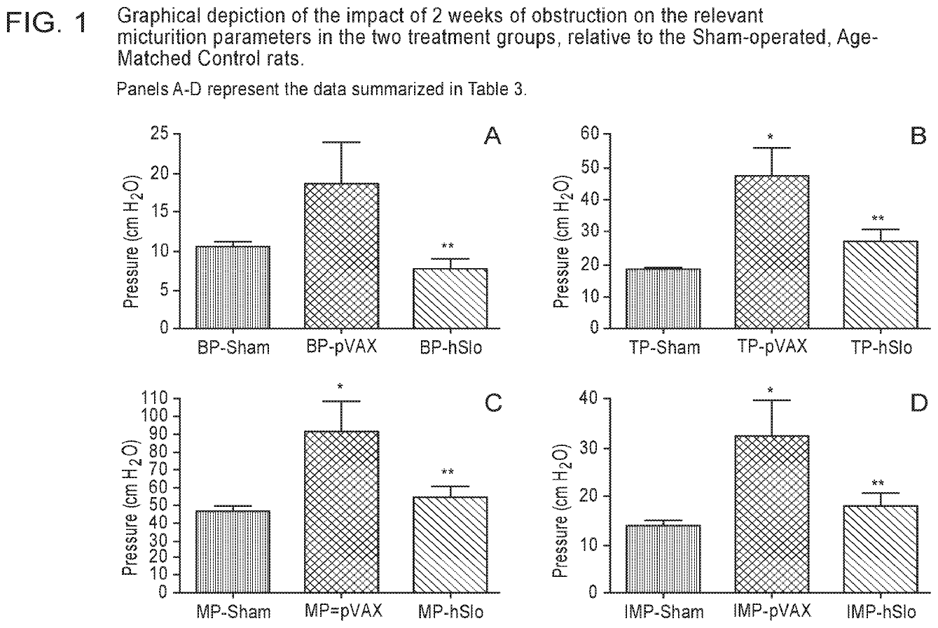

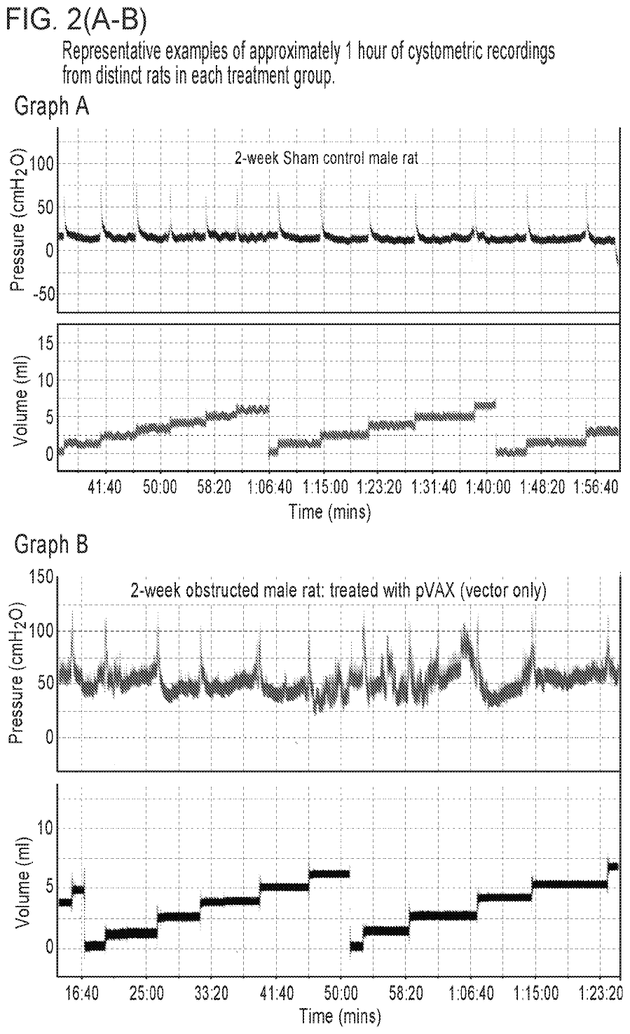

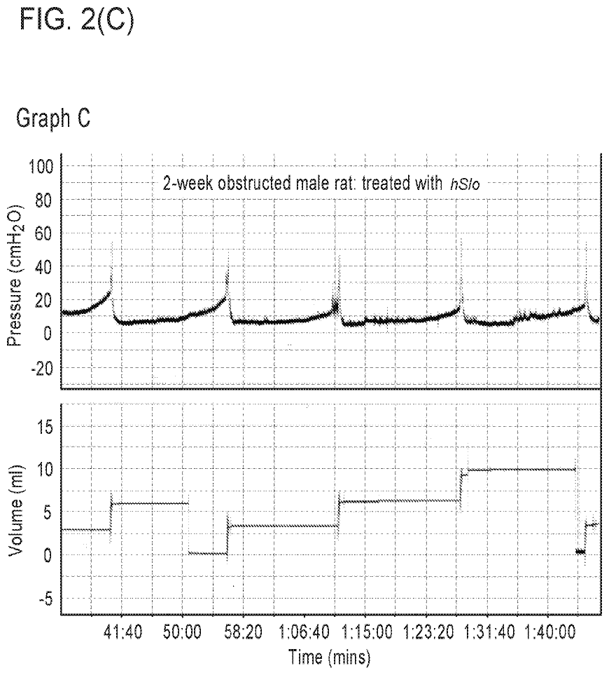

The mean values for the micturition parameters in all experimental animals are summarized in Table 2, and the salient features of these findings are graphically depicted in FIGS. 1 and 2. Importantly, as with the study in the 6-week obstructed female rat a single intravesical instillation of 100 μg hSlo/pVAX was associated with statistically significant changes in several micturition parameters of major physiological relevance.

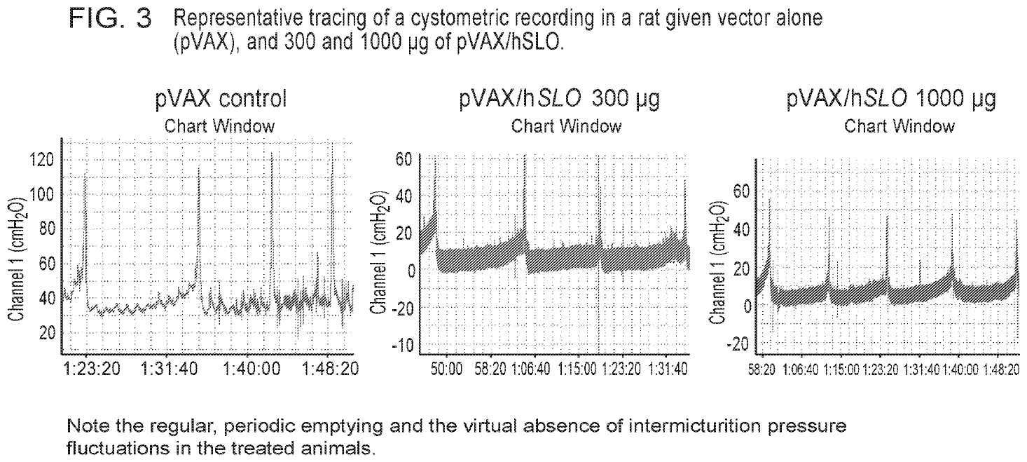

A third study evaluated the effects of hSlo gene transfer following 2 weeks of partial urethral outlet obstruction in female rats. In order to create a partial urethral outlet obstruction (PUO), a ligature was placed on the urethra of female Sprague-Dawley rats weighing 200-250 g (Christ et al., 2001) as described above. Two weeks after placement of the ligature, the rats were subjected to surgery for placement of a suprapubic catheter. Two days later, bladder function studies (i.e., cystometry) were performed on conscious, unrestrained rats in metabolic cages. As illustrated in Table 3 and FIG. 3, following the 2 weeks of partial urethral outlet obstruction female rats exhibit significant changes in bladder function, as evidenced by the more than 2-fold increase in bladder capacity and the appearance of significant spontaneous bladder contractions. The increased spontaneous bladder contractions were observed as pressure fluctuations between micturitions (see FIG. 3), and can be quantified as shown in Table 3 by the corresponding increases observed in the SA and IMP values. A single intraluminal bladder injection of 300 μg and 1000 μg of pVAX/hSlo (in 1 ml PBS-20% sucrose) resulted in a nearly complete ablation of detrusor overactivity. This effect is reflected by the significant decrease in IMP and SA in the hSlo-treated, obstructed rats when compared with the rats treated with pVAX vector only (see Table 3). Although, a true DO effect relationship for hSlo gene transfer was not shown in this model, this study did demonstrate that over a 1-log unit variation in DO (from 100 to 1000 μg), there is a statistically significant, and moreover, physiologically relevant, diminution in DO, in the absence of any detectable effect on the ability of the bladder to empty. That is, in this animal model, pVAX/hSlo is able to ameliorate the pathophysiological effects of outflow obstruction-related DO, without having any detrimental effect on bladder function. Similar effects were observed after instillation of 100 g pVAX/hSlo in the 6-week obstructed female Sprague Dawley rats, which are shown below.

A rabbit study to evaluate the distribution of different volumes of gene transfer injected into the bladder wall was performed prior to initiation of the clinical trial in women with OAB using direct intravesicular injections (Table 4). Nine female Adult New Zealand white rabbits weighing an average of 6 pounds were used. The animals were anesthetized and pVAX-lacz was to be injected into the detrusor in 0.05, 0.1, and 0.15 ml aliquots into 4, 8, and 10 sites in the bladder wall. An additional set of 3 animals was to be injected with carrier alone at only the highest volume of carrier (4, 8, or 10 sites×0.15 ml). The plasmids were in solution at a concentration of 4000 μg/ml. One week later the animals were euthanized and the bladders excised and weighed. Areas with blue color were prepared for histological examination and molecular analysis. Molecular analysis of hSlo expression tissue was done with RNA extraction and real time PCR. In addition, histopathology was performed on the various rabbit tissues.

Due to difficulty with direct bladder injections in this animal model, only one rabbit was given the 0.05 ml injection. Six rabbits had 0.1 ml at 4, 8, and 10 sites (3 from inside the bladder; 3 from outside the bladder). Three rabbits had 0.15 ml at 4, 8, and 10 sites. Results indicated that those rabbits with a greater number of injections (8-10 injections) had less expression than some animals with the smallest number of injections (4 injections). The overall conclusion is that the direct injection into the bladder wall results in expression of the gene, however, it seems to work best with wider dispersion of the injections perhaps 1 cm apart. The gene was detected in the blood up until 30 minutes post treatment. There were granulomatous lesions observed due to the sutures (a common artifact in the rabbit model).

### Toxicology

For the OAB indication it is not technically possible to simulate the same transurethral route of intravesical administration of pVAX/hSlo in rats as will be used in the human trials. Therefore, in the toxicology and biodistribution studies evaluating intravesical injection of pVAX/hSlo, animals underwent surgical exposure of the bladder and study material was injected directly into the bladder using a needle

The effect of pVAX/hSlo on hematological and chemical parameters were assessed in fifteen 275-300 gm normal female Sprague-Dawley rats. 1000 μg of either pVAX/hSlo (8 animals) or pVAX vector (7 animals) was injected directly into the lumen of the bladder following surgical exposure. Blood samples were collected via a heart stick immediately after the animals were euthanized by CO₂ anesthesia at 4, 8, and 24 hours and at 1 week following injection of test material. Samples were analyzed for glucose, urea nitrogen, creatinine, total protein, total bilirubin, alkaline phosphatase, ALT, AST, cholesterol, sodium, potassium, chloride, A/G ratio, BUN/creatinine ratio, globulin, lipase, amylase, triglycerides, CPK, GTP, magnesium and osmolality. The laboratory parameters were similar between pVax/hSlo and controls at the four timepoints.

The effect of pVAX/hSlo on the histopathology in female Sprague-Dawley rats (275 to 300 gm) was evaluated in two studies. In the first study, four rats underwent partial bladder obstruction surgery and 2 weeks later 100 μg pVAX/hSlo in 1000 μL PBS-20% sucrose was administered directly into the lumen of the bladder with surgical exposure of the bladder. A single animal was euthanized at 1, 8, and 24 hours, and at one week after injection of pVAX/hSlo. The tissues of 47 organs were immediately fixed in 10% formalin and processed for routine histopathological examination. Histopathological changes were noted only in the bladder and consisted of serositis, edema, hemorrhage, and fibrosis. These changes were consistent with those expected with partial urethral obstruction and were not considered related to injection of pVAX/hSlo.

Because of the histopathological changes in the bladder of rats with PUO administered pVAX/hSlo, the effect of pVAX/hSlo compared to vector (pVAX) and PBS-20% sucrose on histology of the bladder was evaluated in normal rats. Following surgical exposure, the following test material was injected directly into the bladder lumen: 1) 0.6 ml PBS-20% sucrose, 2) 1000 μg pVAX in 6.6 ml PBS-20% sucrose, or 3) 1000 μg pVAX/hSlo in 0.6 ml PBS-20% sucrose. Animals were euthanized with CO₂ 72 hours after instillation and the bladders removed and immediately fixed in 10% formalin solution. The 72 hour time point was chosen to limit the mechanical effects of the needle puncture on the bladder wall and minimize any potential effects of inflammation that might be caused by the pVAX/hSlo, vector, or diluent.

There were no gross findings on examination of the bladder. Overall, there were no treatment-related differences between pVAX/hSlo and either the vehicle or pVAX. No treatment-related alterations in the urothelium were noted. The lesions seen on histological examination were consistent with trauma from the needle used for injection since they were focal rather than diffuse or multifocal in distribution.

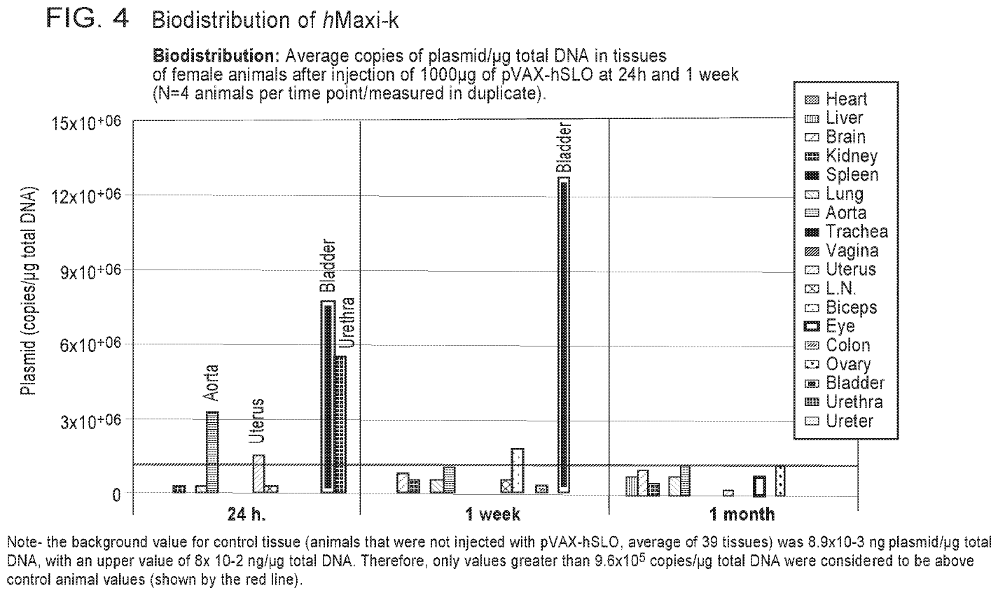

In the biodistribution study, test material was injected directly into the lumen of exposed bladders in 275-300 g normal female Sprague-Dawley rats. 1000 μg pVAX/hSlo in 0.6 ml of PBS-20% sucrose was administered to 12 animals and 0.6 ml PBS-20% sucrose administered to 5 animals (FIG. 4). Four animals each were sacrificed at 24 hours, 1 week, and 1 month following injection of test material. Tissue samples were collected in the specified order as follows: heart, liver, brain, kidney, spleen, lung, aorta, trachea, lymph node, eye, biceps, colon, vagina, and uterus.

Genomic DNA samples were analyzed for the kanamycin gene with a validated QPCR method. The results indicate that after injection of 1000 μg pVAX/hSlo, the plasmid could be detected after 24 hours in the aorta, uterus, bladder, and urethra. At 1 week, approximately 13 million copies/μg total DNA were measured in the bladder and pVAX/hSlo could also be detected slightly in the biceps. The results are displayed in graphical format in FIG. 4 (below).

Although these results differ from findings after intracavernous injection, the detection of 13 million copies/μg total DNA is still lower than the <30 copies plasmid/10⁵ host cells that persist at the site of DNA vaccine injections after 60 days in clinical Investigational New Drug (IND) trials for these vaccines. These DNA vaccine studies have demonstrated that intramuscular, subcutaneous, intradermal, or particle-mediated delivery did not result in long-term persistence of plasmid at ectopic sites. In addition, the procedure to inject pVAX/hSlo directly into the surgically exposed bladder in animals may explain the ability to detect plasmid in tissue other than the bladder. In humans, hMaxi-K will be instilled directly into the bladder using a using a transurethral catheter and the risk of plasmid distribution due to tissue damage or trauma is obviously markedly reduced.

### Example 2: Human Clinical Trial with of HMAXI-K Gene Transfer

### Trial Design

This is a Phase 1B, multicenter study evaluating the safety and potential activity of two escalating doses of hMaxi-K gene administered as a direct Injection into the bladder wall in female patients with Idiopathic (Non-neurogenic) Overactive Bladder Syndrome (OAB) and Detrusor Overactivity (DO).

The study population is women ≥18 years old of non-child bearing potential (e.g., hysterectomy, tubal ligation or postmenopausal defined as last menstrual cycle >12 months prior to study enrollment, or serum FSH>40 mIU/L) with overactive bladder (OAB) and detrusor overactivity who are otherwise in good health.

Inclusion criteria include clinical symptoms of overactive bladder of ≥6 months duration including at least one of the following:
1. Frequent micturition (≥8/24 hrs)
2. Symptoms of urinary urgency (the complaint of sudden compelling desire to pass urine, which is difficult to defer) or nocturia (the complaint of waking at night two or more times to void)
3. Urge urinary incontinence (average of 5 per week—Urge urinary incontinence is defined as: the complaint of involuntary leakage accompanied by or immediately preceded by urgency)

Participants also had a bladder scan at screening demonstrating a residual volume of ≤200 ml and detrusor overactivity documented during baseline urodynamic testing of ≥1 uncontrolled contraction(s) of the detrusor of at least 5 cm/H₂O.

Table 6 shows an overview of the treatment schedule and procedures by visit.

The primary objective of this study is to evaluate occurrence of adverse events and their relationship to a single treatment of approximately 20 to 30 bladder wall intramuscular injections of hMaxi-K compared to placebo (PBS-20% sucrose). This was a double blind, imbalanced placebo controlled sequential dose trial. Participants were healthy women of 18 years of age or older, of non-childbearing potential, with moderate OAB/DO of ≥six months duration with at least one of the following: frequent micturition≥8 times per day, symptoms of urinary urgency or nocturia (the complaint of waking at night two or more times to void), urge urinary incontinence (five or more incontinence episodes per week), and detrusor overactivity with ≥1 uncontrolled phasic contraction(s) of the detrusor of at least 5 cm/H₂O pressure documented on CMG. All of the participants had failed prior treatment with anticholinergics. Four had failed onabotulinumtoxinA therapy.

Participants were randomly assigned to either hMaxi-K at one of two doses (16,000 μg, or 24,000 μg), or placebo. Treatment was administered as 20-30 IM injections into the bladder wall during cystoscopy. Participants were seen 8 times within a 24-week period with a study follow-up of 18 months. All reported adverse events occurring after study drug dosing were recorded. Complex CMG's were done at screening visit 1A (week—1) and at week 4 (visit 5) and week 24 (visit 8) post-injection. Post void residual volume (PVR) was measured at every visit with a Bladderscan®.

The data to assess efficacy were evaluated using summary descriptive statistics by treatment group (combined placebo vs 2 active treatment groups and combined placebo vs combined treatment groups). Linear mixed effect models were used to estimate difference of changes from baseline between placebo and active treatment and to test whether there was dose-response for different outcomes. Generalized estimating equation (GEE) models were to be used to estimate effects for the binary endpoints.

There were 6 participants who received 16000 μg, 3 participants who received 24000 μg and 4 participants who received placebo. In both active treatment groups, the majority of adverse events (AEs) were mild in severity and all were considered unrelated to the study drug. Two women had mild unrelated UTIs post-treatment with hMaxi-K: one receiving 24000 μg at month after dosing and the other receiving 16000 μg at 6 months after dosing. There was one unrelated serious AE reported in the 16000 μg group; exacerbation of pre-existing asthma due to the cold weather which required an ER visit and resolved after asthma treatment was given. No subject was discontinued due to an AE and all enrolled subjects completed the 6 month trial. In addition, during the 18 month long-term post study safety follow-up, no issues were reported in the subjects followed to date (9 of 13 completed 18 month follow-ups; 13 of 13 completed the 12 month follow-ups).

The average of diary data collected for 7 days prior to each visit revealed statistically significant (p<0.05) improvements vs placebo and baseline with durable reduction in mean number of voids per day and mean number of urgency episodes per day over the 6 months of the trial. The changes displayed in tables 7 and 8 below are mean changes (+/−SE) from baseline compared to placebo.

Quality of life parameters (King Health Questionnaire) showed statistically significant sustained mean changes for the individual active treatments and for the combined active treatment groups (all doses) vs placebo and vs baseline in the domains of Impact on Life, Role Limitations, Physical Limitations, Social Limitations and Sleep Energy.

Results from this phase IB clinical trial showed a significant reduction of the number of voiding and urgency episodes after a single administration of hMaxi-K lasted for the 6 month duration of the trial. Those results were observed in the absence of a change in PVR and treatment-related serious adverse events. The results of this novel clinical trial show for the first time that a single intradetrusor administration of human Maxi-K gene was safe.

Despite the small population enrolled, overall findings from the participant diaries showed significant reductions (p<0.05) for the mean number of voids and mean number of urgency episodes vs placebo and vs. baseline for all active treatments and of urge incontinence episodes vs baseline for all doses of study drug. Participant response to treatment showed some positive p values for all active doses vs placebo at Visits 3 and 5 (see Table 9). For the reduction in number of voids and urgency episodes, these significant changes vs placebo and vs baseline were seen at all visits out to final Visit 8 (24 weeks). There were no significant differences seen between the 2 active treatments (16000 μg and 24000 μg) possibly due to the small number of participants enrolled in the 24000 μg group (N=3).

Quality of life parameters (King Health Questionnaire) showed statistically significant mean improvement for the individual active treatments and for the combined active treatment groups (all doses) vs placebo and vs baseline in many of the domains. This included the following:
Domain 2: Impact on Life
   P=0.014 for all active doses and p=0.007 for 24000 μg at Visit 5 vs baseline,
   P=0.016 for 24000 μg at Visit 5 vs placebo;
   P=0.016 for the 24000 μg group vs 16000 μg group at Visit 5
   P=0.043 for all active doses vs baseline at Visit 6
   P=0.010 for 16000 μg and p=0.005 for all active doses vs baseline at Visit 7
   P=0.026 for all active doses vs baseline at Visit 8
Domain 3: Role Limitations
   P=0.004. P=0.015, P<0.001 for 16000 μg, 24000 μg and all active doses, respectively, vs baseline at Visit 5
   P=0.030, P=0.035 and P=0.015 for 16000 μg, 24000 μg and all active doses, respectively, vs placebo at Visit 5
   P=0-023, P=0.014 and P=0.001 for 16000 μg, 24000 μg and all active doses, respectively, vs baseline at Visit 6
   P=0.047, P=0.020 and P=0.014 for 16000 μg, 24000 μg and all active doses, respectively, vs placebo at Visit 6
   P=0.012, P=0.014 and P<0.001 for 16000 μg, 240000 μg and all active doses, respectively, vs placebo at Visit 7
   P=0.032 and P=0.021 for 24000 μg and all active doses, respectively, vs placebo at Visit 7
   P=0.014 and P=0.005 for 24000 μg and all active doses, respectively, vs baseline at Visit 8
   P=0.047. P=0.007 and P=0.007 for 16000 μg, 24000 μg and all active doses, respectively, vs placebo at Visit 8
Domain 4 Physical Limitations
   P=0.018 and P=0.005 for 24000 μg and all active doses, respectively, vs baseline at Visit 6
   P=0.012, P=0.018 and P=0.001 for 16000 μg, 24000 μg and all active doses, respectively, vs baseline at Visit 7
   P=0.012, P=0.047 and P=0.003 for 16000 μg, 24000 μg and all active doses, respectively, vs. baseline at Visit 8
Domain 5: Social Limitations
   P=0.032 and P=0.22, for 24000 μg vs baseline and placebo, respectively, at Visit 6
   P=0.002 and P=0.004 for 24000 μg and all active doses, respectively, vs baseline at Visit 7
   P=0.008 and P=0.043 for 24000 μg and all active doses, respectively, vs placebo at Visit 7
   P=0.002 and P=0.014 for 24000 μg and all active doses, respectively, vs baseline at Visit 8
   P=0.006 for 24000 μg vs placebo at Visit 8
Domain 8: Sleep Energy
   P=0.047 P=0.007 and P=0.001 for 16000 μg, 24000 μg and all active doses, respectively, vs baseline at Visit 5
   P=0.020 and P=0.015 for 24000 μg and all active doses, respectively, vs placebo at Visit 5
   P=0.005 and P=0.006 for 24000 μg and all active doses, respectively, vs baseline at Visit 6
   P=0.001 and P=0.006 for 24000 μg and all active doses, respectively, vs baseline at Visit 7
   P=0.012 for 24000 μg vs placebo at Visit 7

The 72 hour Pad Test (Table 12) showed some statistically significant changes at Visit 3-6 and Visit 8 for hMaxi-K active doses vs baseline, however, there were also statistically significant changes for placebo at Visits 3-5 and Visit 8. Overall the placebo group appeared to have less severe disease than the active treatment groups with baseline (V2) pad weights for active treatment being almost 2 times greater than that of the placebo group. In addition, the V1A mean pad weight for placebo was only 29 grams whereas the weight at V2 for this group was 259 grams (almost 9 times greater than V1A). This was due to the fact that participant 002-001 had thrown out her pads prior to V1A (so she was not included in the V1A means) and she appears to have had more severe disease than the other 3 placebo participants (her 3-day average pad weight at V2 was 295 grams vs 3.3 to 36 grams for the other 3 participants).

TABLE 1
Summary of treatment effects on mean micturition parameters in 6 week obstructed female rats and sham-operated controls

| | WT | | | | | | | MIP |
|---|---|---|---|---|---|---|---|---|
| | (mg) | MP | THP | BP | BC | MV | RV | (IP − BP) |
| Control: | 171 ± 15.0 | 73.9 ± 4.99 | 22.3 ± 2.1 | 12.6 ± 1.09 | 1.2 ± 0.1 | 1.13 ± 0.10 | 0.13 ± 0.04 | 3.49 ± 0.79 |
| unobstructed | | | | | | | | |
| (n = 17) | | | | | | | | |
| ^{a}Obstructed: | *547.6 ± 55.4 | *128.9 ± 16.1 | *36.3 ± 4.30 | *22.1 ± 43.9 | *3.44 ± 0.41 | *3.22 ± 0.39 | *^{,}**0.3 ± 0.10 | **5.59 ± 1.05 |
| pVAX/hSlo | | | | | | | | |
| injected | | | | | | | | |
| (n = 12) | | | | | | | | |
| ^{b}Obstructed: | *473.1 ± 56.6 | *132.7 ± 17.9 | *39.3 ± 3.6 | *18.8 ± 1.9 | *2.91 ± 0.62 | *2.94 ± 0.65 | 0.09 ± 0.05 | 9.37 ± 1.79 |
| untreated | | | | | | | | |
| (n = 10) | | | | | | | | |

^{a}100 μg pVAX/hSlo in 200 μl PBS-20% sucrose
^{b}3 of these rats received 1000 μg pcDNA in PBS-20% sucrose. Control: Sham operated, unobstructed age-matched control animals, WT: bladder weight (mg), MP: micturition pressure (cm H₂O). THP: threshold pressure (cm H₂•), BP: basal pressure (cm H₂•), BC: bladder capacity (ml), MV: micturition volume (ml). RV: residual volume (ml). MIP: mean inter-micturition pressure ((cm H₂O; the mean pressure over the entire inter-micturition interval minus the basal pressure on the same animal).
*Significantly different from sham-op; p < 0.05.
**Significantly different from control (obstructed but not treated); p < 0.05, One-Way ANOVA, with Newman Keuls post hoc pairwise comparisons.

TABLE 2
Summary of treatment effects on mean micturition parameters
in 2 week obstructed male rats and sham-operated controls.

| | Bcap | MV | RV | BP | TP | MP |
|---|---|---|---|---|---|---|
| pVAX | 2.36 ± 0.48 | 1.84 ± 0.31 | 0.53 ± 0.21 | 18.65 ± 5.38 | 47.21 ± 8.61^{c} | 91.28 ± 18.52^{c} |
| (n = 8)^{b} | | | | | | |
| hSlo | 2.48 ± 0.30c | 2.22 ± 0.26^{c} | 0.27 ± 0.12 | 7.66 ± 1.35^{d} | 27.26 ± 3.7^{d} | 54.05 ± 6.28^{d} |
| (n = 16)^{b} | | | | | | |
| Sham | 1.35 ± 0.14 | 1.32 ± 0.12 | 0.03 ± 0.02 | 10.6 ± 0.81 | 18.47 ± 0.79 | 46.58 ± 3.34 |
| (n = 10)^{a} | | | | | | |

| | IMP | SA | Bcom | BW |
|---|---|---|---|---|
| pVAX | 32.49 ± 7 5^{c} | 13.84 ± 2.57^{c} | 0.12 ± 0.04 | 348.3 ± 105.3 |
| (n = 8)^{b} | | | | |
| hSlo | 18.13 ± 2.8^{d} | 10.47 ± 1.89^{c} | 0.17 ± 0.03 | 352.3 ± 42.99 |
| (n = 16)^{b} | | | | |
| Sham | 13.96 ± 1.09 | 3.39 ± 0.61 | 0.18 ± 0.018 | 274.4 ± 24.5 |
| (n = 10)^{a} | | | | |

Bcap, bladder capacity (ml); MV, micturition volume (ml); RV, residual volume (ml); BP, basal pressure (cm H₂O); TP. threshold pressure (cm H₂O); MP, micturition pressure (cm H₂O); IMP, mean intermicturition pressure (cm H₂O; the mean pressure over the entire intermicturition interval minus the basal pressure on the same animal); SA, spontaneous activity (cm H₂O); Bcom, bladder compliance (ml/cm H₂O); BW, bladder weight (mg).
^{a}5 of these animals are 2-week sham controls, the other 5 are 1 month older (or 6-week sham controls). However, statistical analysis revealed that there were no significant differences in any of the micturition parameters, and thus, these 2 populations were considered to be homogeneous for the purposes of this analysis
^{b}All treated rats were given 1000 μg pVAX alone or 100 μg hSlo/pVAX in 1 ml PBS with 20% sucrose. All data represent the mean ± S.E.M. and were analyzed using a one-way analysis of variance, with a post hoc Tukey's test for all pairwise (multiple) comparisons.
^{c}Significant difference from the corresponding sham control value.
^{d}Significant difference from the corresponding pVAX value.

TABLE 3
Summary of treatment effects on mean micturition parameters in 2 week obstructed female rats

| | MP | TP | BP | BC | MV | RV |
|---|---|---|---|---|---|---|
| Control: pVAX | 68.1 ± 8.1 | 34.2 ± 4.9 | 9.1 ± 1.9 | 2.3 ± 0.3 | 2.2 ± 0.3 | 1.1 ± 0.0 |
| (n = 10) | | | | | | |
| ^{a}Obstructed: 10 | 65.3 ± 10.5 | 30.3 ± 3.6 | 7.2 ± 1.0 | 2.5 ± 0.3 | 2.4 ± 0.3 | 0.2 ± 0.1 |
| μg pVAX/hSlo | | | | | | |
| injected (n = 7) | | | | | | |
| ^{b}Obstructed: 30 | 81.1 ± 7.3 | 36.6 ± 4.4 | 11.8 ± 2.6 | 3.2 ± 1.0 | 2.7 ± 0.4 | 0.4 ± 0.2 |
| μg pVAX/hSlo | | | | | | |
| injected (n = 9) | | | | | | |
| ^{b}Obstructed: 300 | 47.8 ± 3.7 | 17.7*,** ± 1.6 | 6.3 ± 1.1 | 2.3 ± 0.4 | 2.2 ± 0.3 | 0.3 ± 0.2 |
| μg pVAX/hSlo | | | | | | |
| injected (n = 10) | | | | | | |
| ^{b}Obstructed: | 57.2 ± 6.2 | 21.4*,** ± 1.8 | 5.7 ± 1.1 | 2.1 ± 0.1 | 2.0 ± 0.1 | 0.1 ± 0.04 |
| 1000 μg | | | | | | |
| pVAX/hSlo | | | | | | |
| injected (n = 12) | | | | | | |

| | MIP | | | |
|---|---|---|---|---|
| | (IP − BP) | MF | SA | BCOM |
| Control: pVAX | 24.0 ± 4.6 | 4.6 ± 0.5 | 14.9 ± 3.4 | 0.1 ± 0.02 |
| (n = 10) | | | | |
| ^{a}Obstructed: 10 | 20.0 ± 3.5 | 4.4 ± 0.5 | 12.8 ± 3.0 | 0.1 ± 0.02 |
| μg pVAX/hSlo | | | | |
| injected (n = 7) | | | | |
| ^{b}Obstructed: 30 | 27.1 ± 3.5 | 4.3 ± 0.4 | 15.3 ± 1.5 | 0.1 ± 0.02 |
| μg pVAX/hSlo | | | | |
| injected (n = 9) | | | | |
| ^{b}Obstructed: 300 | 10.3*,** ± 1.2 | 5.3 ± 0.6 | 4.1*,** ± 0.4 | 0.2*,** ± 0.02 |
| μg pVAX/hSlo | | | | |
| injected (n = 10) | | | | |
| ^{b}Obstructed: | 11.6*,** ± 1.3 | 5.2 + 0.3 | 5.9*,** ± 0.5 | 0.1*,** ± 0.01 |
| 1000 μg | | | | |
| pVAX/hSlo | | | | |
| injected (n = 12) | | | | |

^{a}10, 30, 300, 1000 μg pVAX/hSlo in 200 μl PBS-20% sucrose
^{b}Control: Obstructed age-matched control animals that received 1000 μg of pVAX only, WT; bladder weight (mg), MP: micturition pressure (cm H₂O), TP: threshold pressure (cm H₂O), BP: basal pressure (cm H₂O), BC: bladder capacity (ml), MV: micturition volume (ml), RV: residual volume (ml), MIP: mean inter-micturition pressure ((cm H₂O; the mean pressure over the entire inter-micturition interval minus the basal pressure on the same animal); SA spontaneous activity (MIP − BP): BCOM Bladder compliance (bladder capacity/TP − BP)
*Significantly different from control; p < 0.05. All pairwise multiple comparison procedures (Holm-Sidak method)
Significantly different from control; p < 0.05, One-Way ANOVA.

TABLE 4
Rabbit Intravesicular Injection Protocol

| | 50-50 mixture of | | | |
|---|---|---|---|---|
| | p-VAXhslo (ml) | sites/rabbit | sites/rabbit | sites/rabbit |
| N = 12 | 0.05 | 4 | 8 | 10 |
| rabbits | 0.1 | 4 | 8 | 10 |
| | 0.15 | 4 | 8 | 10 |

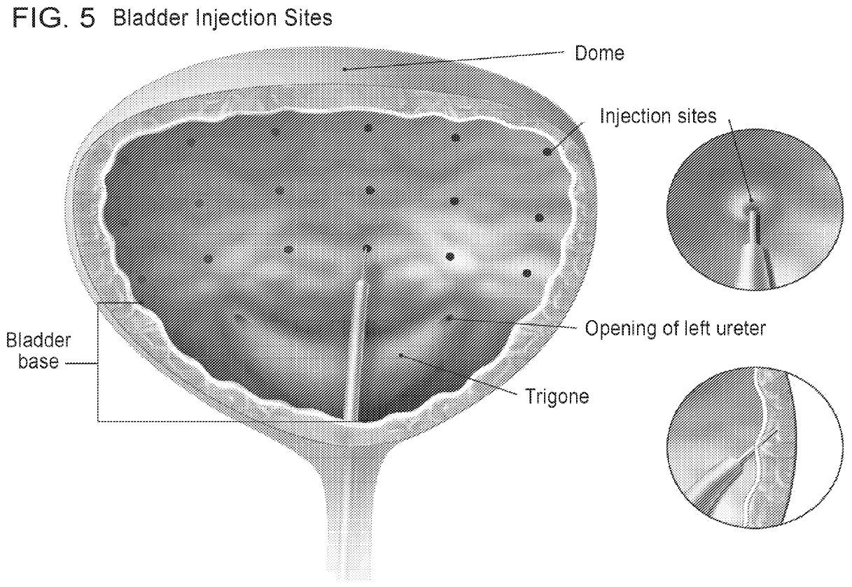

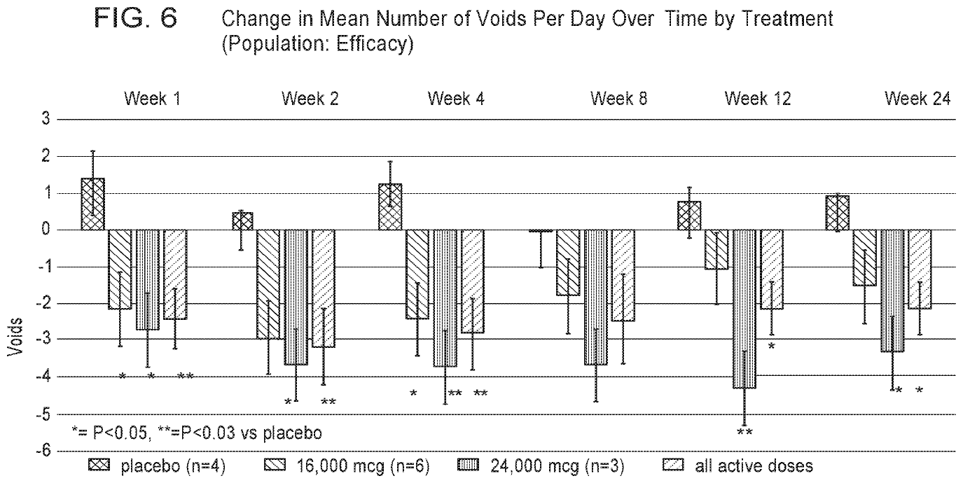

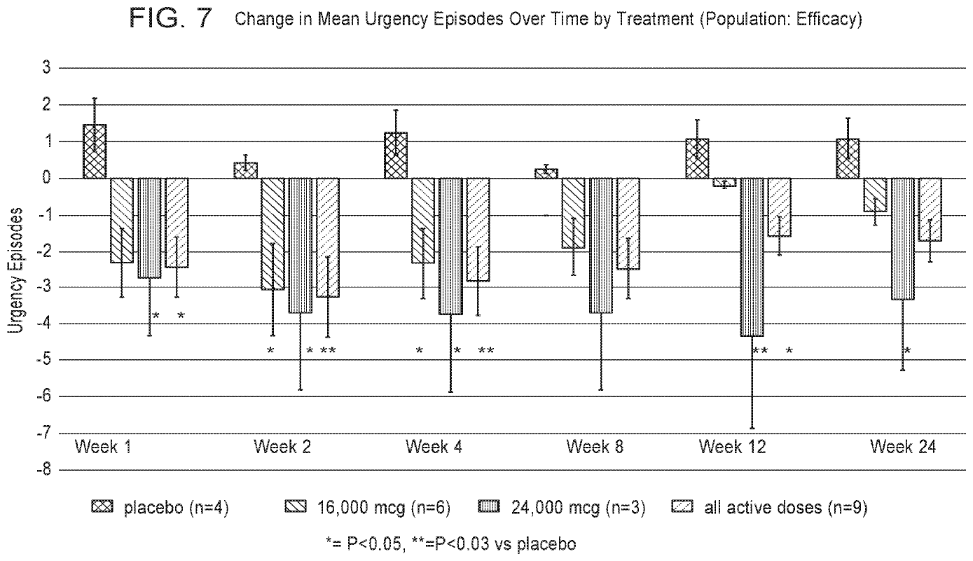

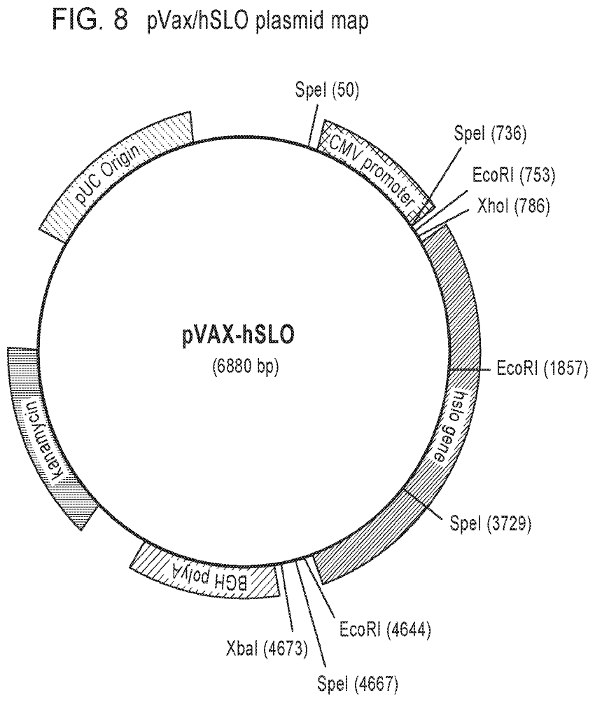

TABLE 5
Final Dose-hMaxi-k

| | | | | |
|---|---|---|---|---|
| hMaxi-K Dose | 16,000 μg | PBS-20% sucrose | 24,000 μg | PBS-20% sucrose |

| | | |
|---|---|---|
| Volume | 4 mL | 6 mb |
| Number of Vials | 2 | 3 |
| Final Volume | 4 mb | 6 mb |
| Number of IM | 20 injections of 0.2 ml at specified | 30 injections of 0.2 ml at specified sites |
| injections | sites in bladder wall approx. 1 cm apart | in bladder wall approx. 1 cm apart |
| | (FIG. 5) | (FIG. 5) |

Note:
In each dose cohort 6 participants will receive hMaxi-K and 3 will receive PBS-20% sucrose (placebo).

TABLE 6
Summary of Tests by Laboratory Visit

| | Phase | | | | | | | | | |
|---|---|---|---|---|---|---|---|---|---|---|
| | Screening Phase | | | Post-Treatment Follow up Visits | | | | | | |
| | Visit/Period | | | | | | | | | |
| | | | | Telephone | | | | | | |
| | Visit 1 | Visit 1Aⁿ | Visit 2 | Follow-upⁱ | Visit 3 | Visit 4 | Visit 5 | Visit 6 | Visit 7 | Visit 8 |
| | Day | | | | | | | | | |
| | −14 | −14 to −3 | 0 (Baseline) | Day 1 & 3 | 8 | 15 | 29 | 57 | 35 | 169 (Final) |
| | Week | | | | | | | | | |
| | −2 | | 0 | 0 | 3 | 2 | 4 | 3 | 12 | 24 |
| | Visit Window (days) | | | | | | | | | |
| | | +2 | +2 | Day 3 ± 1 | +2 | +2 | ±2 | ±3 | ±5 | ±5 |
| Signed Informed Consent | ▴ | | | | | | | | | |
| Evaluation of Inclusion/ | ▴ | ▴ | ▴ ^{f} | | | | | | | |
| Exclusion Criteria | | | | | | | | | | |
| Demographics and Medical/ | ▴ | | | | | | | | | |
| Surgical History | | | | | | | | | | |
| Physical Examination | ▴ | | ▴ ^{f} | | ▴ | ▴ | ▴ | ▴ | ▴ | ▴ |
| ECG | ▴ | | ▴ ⁰ | | ▴ | | ▴ | | | ▴ |
| Previous/Concomitant | ▴ | ▴ | ▴ ^{f} | | ▴ | ▴ | ▴ | ▴ | ▴ | ▴ |
| Medication Assessment | | | | | | | | | | |
| Vital Signs ^{h} | ▴ | | ▴ ^{f, l} | | ▴ | ▴ | ▴ | ▴ | ▴ | ▴ |
| Objective OAB/DO | | ▴^{d} | | | | | ▴ | | | ▴ |
| Evaluation (Cystometry) ^{b} | | | | | | | | | | |
| Bladder scan^{c} | ▴ | | | | | ▴ | | ▴ | | ▴ |
| Dispense Daily Voiding | ▴ | ▴ | ▴ ^{f} | | ▴ | ▴ | ▴ | ▴ | ▴ | |
| Diary/Urgency questionnaire ^{l} | | | | | | | | | | |
| Pad Test ^{m} | | ▴ | ▴ | | ▴ | ▴ | ▴ | ▴ | ▴ | ▴ |
| Qol. (King's Health | | | ▴ ^{f} | | | | ▴ | ▴ | ▴ | ▴ |
| Questionnaire) and SF-12 | | | | | | | | | | |
| Subjective Evaluation | | | ▴ ^{f} | | ▴ | ▴ | ▴ | ▴ | ▴ | ▴ |
| of Disease State ^{k} | | | | | | | | | | |
| Subjective Evaluation | | | | | ▴ | ▴ | ▴ | ▴ | ▴ | ▴ |
| of Response to Treatment ^{k} | | | | | | | | | | |
| ICIQ-SF | | | ▴ ^{f} | | | | ▴ | ▴ | ▴ | ▴ |
| Urinalysis and Urine | ▴ | ▴ | ▴ ^{f} | ▴ | ▴ | ▴ | ▴ | | ▴ | ▴ |
| Cultures ^{d} | | | | | | | | | | |
| Hematology | ▴ | | ▴ | ▴ | ▴ | ▴ | ▴ | | ▴ | ▴ |
| Laboratory Tests^{e} | | | | | | | | | | |
| Chemistry Laboratory | ▴ | | ▴ | ▴ | ▴ | ▴ | ▴ | | ▴ | ▴ |
| Tests^{e} | | | | | | | | | | |
| Pharmacokinetic Assessment | | | ▴ ^{f, g} | | ▴ | ▴ | ▴ | ▴ | ▴ | ▴ ^{g} |
| (urine and blood hSlo cDNA) | | | | | | | | | | |
| Adverse Event Assessment | | ▴ | ▴ ^{f} | ▴ ^{f} | ▴ | ▴ | ▴ | ▴ | ▴ | ▴ |
| Study Drug administered | | | ▴ | | | | | | | |

^{a} ECG will be done prior to administration of study drug and at 2 hours post dosing.
^{b} Cystometry includes: volume at first desire to void, detrusor pressure, abdominal pressure, detrusor pressure at beginning of voiding, detrusor pressure at maximum flow, maximum detrusor pressure, volume at strong urge to void, peak flow rate during voiding, voided volume, volume at DO, post-void residual volume, total bladder volume (voided volume + residual volume), number of detrusor contractions during procedure and duration of DO.
^{c}Inclusion criteria specify residual volume ≤200 ml. Bladder scans at V1 and V8 to be done before catheterization.
^{d}Urinalysis with microscopic RBC and WBC, protein, glucose, nitrites, pH, and specific gravity at V 1, 3-5 and V7 and V8. At V1A and V2, urinalysis by Dipstick will be done. Urine cultures at V1 (by catheterization with the urodynamic catheter), V3 (clean void); at V1A. V2. V5 and V8 prior to cystometry or cystoscopy (by catheterization with the urodynamic catheter) and before discharge by clean void (at V2 use first voided urine after drug administration). Visit 2 urinalysis by Dipstick will be done prior to dosing and urine culture will be performed both prior to study drug administration and prior to discharge.
^{e}Lab tests to be done at V1, V2-5, V7 and V8 include: Hematology- CBC with differential, platelet count, sedimentation rate, PTT, PT (no PT and PTT at V2 and V4), CRP, Antinuclear antibody; Chemistry- BUN, creatinine, Na⁺, K⁺, Mg⁺⁺, Ca⁺⁺, CO2, Cl⁻, albumin, alkaline phosphatase, ALT, AST, GGT, total bilirubin, total protein, CPK, LDH, glucose); Serum wPregnancy test for beta-HCG required for women of child bearing age who have not had hysterectomy at Screening V1 and on as need basis. In addition, FSH >40 IU/L if last menstrual cycle not >12 months prior to study enrollment. HbA1c will be done at screening Visit 1 only. No chemistries will be done at 2 (Week 0). At V4, chemistries will include only BUN, creatinine, electrolytes (Na⁺, K⁺), CRP, glucose, and ANA. No lab tests will be done at Visit 1A or V6. Lab tests should be taken at the same time of day at all study visits.
^{f} Test or procedure will be done prior to administration of study drug at Visit 2
^{g} Pre-dosing at V2. If specimen is still positive at week 24, participant must return monthly until two successive specimens are negative for hSlo DNA.
^{h} Vital signs will include height at V1 only; weight at V1 and V8; oral body temperature at all visits (except V1A). Same arm should be used for all BP measurements and specified.
ⁱDiaries are to be completed prior to V1A (to test for compliance and inclusion criteria), for 7 days prior to Visit 2 and 7 days prior to each visit, thereafter.
^{j} Participants will be contacted by telephone on Study Day 1 and 3 (1 day and 3 days ± 1. following drug administration at Visit 2) for assessment of adverse events.
^{k} Subjective assessments are based on the following questions in Appendix C: “How bothersome do you consider your bladder problem?” and “Has the treatment been of benefit to you?”
^{l} BP will be taken every 15 minutes for 2 hour post administration of study drug.
^{m} Participants will bring in pads/diapers worn for 3 days prior to Visit 1A & 2 (if V1A after screening V1) and 3 days prior to all subsequent visits (Visit 3 to Visit 8); also bring in clean pad/diapers to use as baseline.
ⁿVisit 1A may occur on same day as V1. In this case all V1A procedures not already to be done at V1 should be completed. Cystoscopy should be performed after all other V1 procedures and post cystoscopy urine culture obtained using clean void. If V1A coincides with V1, then since pad collection and diaries will not be completed prior to V1, these must be checked for compliance at V2.
^{o} ECG will be done prior to administration of study

TABLE 7
Mean Number of Voids/24 Hours and Reduction Over Time - Efficacy Population

| | | hMaxi-K | | | | | | | |
|---|---|---|---|---|---|---|---|---|---|
| | | Placebo | | 16000 ug | | 24000 ug | | All Doses | |
| Visit | n | 4 | | 6 | | 3 | | 9 | |
| Visit 1A | Mean no. voids | 10.46 | (3.48) | 11.99 | (3.65) | 17.39 | (5.22) | 13.79 | (4.73) |
| (Screening) | | | | | | | | | |
| Visit 2 | Mean no. voids | 10.18 | (4.78) | 11.26 | (2.70) | 17.19 | (7.07) | 13.24 | (5.08) |
| (Baseline) | [symbol] | | | | | | | | |
| Visit 3 | Mean no. voids | 11.59 | (4.98) | 9.10 | (2.12) | 14.46 | (3.74) | 10.89 | (3.67) |
| (Week 1) | Mean change | 1.41 | (0.78) | −2.16 | (1.80) | −2.73 | (7.29) | −2.35 | (3.92) |
| | fro tn baseline | | | | | | | | |
| | (SD) | | | | | | | | |
| | SEM | 0.39 | | 0.73 | | 4.21 | | 1.31 | |
| | P-value [1] | 0.251 | | 0.052 | | 0.074 | | 0.018 | |
| | P-value [2] | | | 0.044 | | 0.047 | | 0.027 | |
| | Difference of LS | | | −3.57 | | −4.14 | | −3.86 | |
| | Means vs. | | | | | | | | |
| | 95% CI | | | −7.01, −0.13 | | −8.22, −0.07 | | −7.12, −0.59 | |
| Visit 4 | Mean no. voids | 10.68 | (4.10) | 8.35 | (2.65) | 13.52 | (1.94) | 10.07 | (3.47) |
| (Week 2) | Mean change | 0.51 | (1.22) | −2.92 | (2.04) | −3.67 | (6.48) | −3.17 | (3.64) |
| | from baseline | | | | | | | | |
| | (SD) | | | | | | | | |
| | SEM | 0.61 | | 0.83 | | 3.74 | | 1.21 | |
| | P-value [1] | 0.667 | | 0.016 | | 0.026 | | 0.004 | |
| | P-value [2] | | | 0.051 | | 0.046 | | 0.029 | |
| | Difference of LS | | | −3.42 | | −4.17 | | −3.80 | |
| | Means vs. | | | | | | | | |
| | 95% CI | | | −6.87, 0.02 | | −8.25, −0.10 | | −7.06, −0.53 | |
| Visit 5 | Mean no. voids | 11.40 | (4.42) | 8.87 | (2.25) | 13.48 | (1.08) | 10.40 | (2.96) |
| (Week 4) | Mean change | 1.22 | (0.69) | −2.40 | (2.11) | −3.71 | (7.27) | −2.84 | (4.05) |
| | from baseline | | | | | | | | |
| | (SD) | | | | | | | | |
| | SEM | 0.35 | | 0.86 | | 4.20 | | 1.35 | |
| | P-value [1] | 0.315 | | 0.035 | | 0.025 | | 0.006 | |
| | P-value [2] | | | 0.042 | | 0.024 | | 0.017 | |
| | Difference of LS | | | −3.62 | | −4.93 | | −4.28 | |
| | Means vs. | | | | | | | | |
| | 95% CI | | | −7.06, −0.17 | | −9.01, −0.86 | | −7.54, −1.01 | |
| Visit 6 | Mean no. voids (SD) | 10.17 | (3.89) | 9.48 | (2.73) | 13.52 | (2.19) | 10.83 | (3.15) |
| (Week 8) | Mean change from | −0.01 | (1.20) | −1.79 | (2.15) | −3.67 | (7.75) | −2.41 | (4.33) |
| | baseline (SD) | | | | | | | | |
| | SEM | 0.60 | | 0.88 | | 4.47 | | 1.44 | |
| | P-value [1] | 0.996 | | 0.094 | | 0.026 | | 0.011 | |
| | P-value [2] | | | 0.261 | | 0.071 | | 0.090 | |
| | Difference of LS | | | −1.78 | | −3.66 | | −2.72 | |
| | Means vs. placebo | | | | | | | | |
| | 95% CI | | | −5.22, 1.66 | | −7.74, 0.41 | | −5.99, 0.55 | |
| Visit 7 | Mean no. voids (SD) | 10.96 | (4.30) | 10.21 | (4.11) | 12.90 | (2.35) | 11.11 | (3.71) |
| (Week 12) | Mean change from | 0.79 | (1.67) | −1.05 | (2.90) | −4.29 | (6.97) | −2.13 | (4.47) |
| | baseline (SD) | | | | | | | | |
| | SEM | 0.84 | | 1.18 | | 4.02 | | 1.49 | |
| | P-value [1] | 0.509 | | 0.293 | | 0.013 | | 0.012 | |
| | P-value [2] | | | 0.248 | | 0.022 | | 0.041 | |
| | Difference of LS | | | −1.83 | | −5.07 | | −3.45 | |
| | Means vs. placebo | | | | | | | | |
| | 95% CI | | | −5.28, 1.61 | | −9.15, −1.00 | | −6.72, −0.19 | |
| Visit 8 (Exit | Mean no. voids (SD) | 11.14 | (4.81) | 9.74 | (3.04) | 13.86 | (3.02) | 11.11 | (3.51) |
| Visit - Week 24) | Mean change from | 0.96 | (0.99) | −1.52 | (2.55) | −3.33 | (7.06) | −2.13 | (4.16) |

| | | | | |
|---|---|---|---|---|
| baseline (SD) | | | | |
| SEM | 0.50 | 1.04 | 4.08 | 1.39 |
| P-value [1] | 0.421 | 0.142 | 0.038 | 0.019 |
| P-value [2] | | 0.131 | 0.041 | 0.044 |
| Difference of LS | | −2.49 | −4.30 | −3.39 |
| Means vs. placebo | | | | |
| 95% CI | | −5.93, 0.96 | −8.37, −0.22 | −6.66, −0.13 |

[1]: p-value to test whether there is a statistically significant difference between values measured at certain time point vs baseline measurement for certain treatment.
[2]: p-value for test whether there is a statistically significant difference between changes from baseline comparing to placebo.
All the p-values and estimates are derived from a linear mixed effect model with number of voids as dependent variables, treatments (placebo, 16000 ug, 24000 ug and total hMaxi-K), time point and interaction of time and treatment.
All doses = all hMaxi-K doses
SD = standard deviation;
SEM = standard error of the mean [symbol]
indicates data missing or illegible when filed

TABLE 8
Mean Number of Urgency Episodes/24 Hours and Reduction Over Time - Efficacy Population

| | | hMaxi-K | | | | | | | |
|---|---|---|---|---|---|---|---|---|---|
| | | Placebo | | 16000 ug | | 24000 ug | | All Doses | |
| Visit | n | 4 | | 6 | | 3 | | 9 | |
| Visit 1A | Mean no urgency | 10.04 | (3.80) | 11.12 | (4.08) | 17.27 | (5.33) | 13.17 | (5.19) |
| (Screening) | episodes (SD) | | | | | | | | |
| Visit 2 | Mean no urgency | 9.82 | (5.17) | 10.21 | (3.55) | 17.19 | (7.07) | 12.53 | (5.71) |
| (Baseline) | episodes (SD) | | | | | | | | |
| Visit 3 | Mean no urgency | 11.27 | (5.25) | 7.89 | (3.11) | 14.46 | (3.74) | 10.08 | (4.51) |
| (Week 1) | episodes (SD) | | | | | | | | |
| | Mean change | 1.45 | (0.83) | −2.31 | (2.17) | −2.73 | (7.29) | −2.45 | (4.03) |
| | from baseline | | | | | | | | |
| | (SD) | | | | | | | | |
| | SEM | 0.42 | | 0.88 | | 4.21 | | 1.34 | |
| | P-value [1] | 0.240 | | 0.040 | | 0.074 | | 0.016 | |
| | P-value [2] | | | 0.036 | | 0.046 | | 0.024 | |
| | Difference of LS | | | −3.76 | | −4.18 | | −3.97 | |
| | Means vs. | | | | | | | | |
| | placebo | | | | | | | | |
| | 95% CI | | | −7.20, −0.32 | | −8.25, −0.11 | | −7.23, −0.71 | |
| Visit 4 | Mean no urgency | 10.22 | (4.49) | 7.17 | (3.35) | 13.52 | (1.94) | 9.29 | (4.25) |
| (Week 2) | episodes (SD) | | | | | | | | |
| | Mean change | 0.40 | (1.03) | −3.04 | (2.07) | −3.67 | (6.48) | −3.25 | (3.64) |
| | from baseline | | | | | | | | |
| | (SD) | | | | | | | | |
| | SEM | 0.51 | | 0.85 | | 3.74 | | 1.21 | |
| | P-value [1] | 0.734 | | 0.013 | | 0.026 | | 0.004 | |
| | P-value [2] | | | 0.050 | | 0.050 | | 0.030 | |
| | Difference of LS | | | −3.43 | | −4.07 | | −3.75 | |
| | Means vs. | | | | | | | | |
| | placebo | | | | | | | | |
| | 95% CI | | | −6.87, 0.01 | | −8.14, 0.00 | | −7.01, −0.49 | |
| Visit 5 | Mean no urgency | 11.04 | (4.75) | 7.87 | (3.92) | 13.48 | (1.08) | 9.74 | (4.22) |
| (Week 4) | episodes (SD) | | | | | | | | |
| | Mean change | 1.22 | (0.69) | −2.34 | (2.07) | −3.71 | (7.27) | −2.80 | (4.04) |
| | from baseline | | | | | | | | |
| | (SD) | | | | | | | | |
| | SEM | 0.35 | | 0.84 | | 4.20 | | 1.35 | |
| | P-value [1] | 0.315 | | 0.038 | | 0.025 | | 0.007 | |
| | P-value [2] | | | 0.044 | | 0.024 | | 0.018 | |
| | Difference of LS | | | −3.56 | | −4.93 | | −4.25 | |
| | Means vs. | | | | | | | | |
| | placebo | | | | | | | | |
| | 95% CI | | | −7.00, −0.12 | | −9.00, −0.86 | | −7.51, −0.98 | |
| Visit 6 | Mean no urgency | 9.60 | (4.45) | 8.32 | (4.40) | 13.52 | (2.19) | 10.05 | (4.48) |
| (Week 8) | episodes (SD) | | | | | | | | |
| | Mean change | −0.22 | (0.89) | −1.89 | (2.07) | −3.67 | (7.75) | −2.48 | (4.30) |
| | from baseline | | | | | | | | |
| | (SD) | | | | | | | | |
| | SEM | 0.45 | | 0.85 | | 4.47 | | 1.43 | |
| | P-value [1] | 0.851 | | 0.079 | | 0.026 | | 0.010 | |
| | P-value [2] | | | 0.289 | | 0.085 | | 0.106 | |
| | Difference of LS | | | −1.67 | | −3.45 | | −2.56 | |
| | Means vs. | | | | | | | | |
| | placebo | | | | | | | | |
| | 95% CI | | | −5.11, 1.77 | | −7.52, 0.62 | | −5.82, 0.71 | |
| Visit 7 | Mean no urgency | 10.86 | (4.35) | 10.00 | (4.31) | 12.86 | (2.38) | 10.95 | (3.88) |
| (Week 12) | episodes (SD) | | | | | | | | |
| | Mean change | 1.04 | (2.15) | −0.21 | (2.41) | −4.33 | (7.05) | −1.58 | (4.51) |
| | from baseline | | | | | | | | |
| | (SD) | | | | | | | | |
| | SEM | 1.07 | | 0.99 | | 4.07 | | 1.50 | |
| | P-value [1] | 0.389 | | 0.829 | | 0.013 | | 0.025 | |
| | P-value [2] | | | 0.421 | | 0.017 | | 0.048 | |
| | Difference of LS | | | −1.24 | | −5.37 | | −3.31 | |
| | Means vs. | | | | | | | | |
| | placebo | | | | | | | | |
| | 95% CI | | | −4.68, 2.20 | | −9.44, −1.30 | | −6.57, −0.04 | |
| Visit 8 (Exit | Mean no urgency | 10.89 | (4.99) | 9.29 | (3.53) | 13.86 | (3.02) | 10.81 | (3.91) |
| Visit) (Week 24) | episodes (SD) | | | | | | | | |
| | Mean change | 1.07 | (1.18) | −0.92 | (2.27) | −3.33 | (7.06) | −1.72 | (4.14) |
| | from baseline | | | | | | | | |

| | | | | |
|---|---|---|---|---|
| (SD) | | | | |
| SEM | 0.59 | 0.92 | 4.08 | 1.38 |
| P-value [1] | 0.373 | 0.350 | 0.037 | 0.032 |
| P-value [2] | | 0.213 | 0.038 | 0.054 |
| Difference of LS | | −1.99 | −4.40 | −3.20 |
| Means vs. | | | | |
| placebo | | | | |
| 95% CI | | −5.43, 1.45 | −8.47, −0.33 | −6.46, 0.06 |

[1]: p-value to test whether there is a statistically significant difference between values measured at certain time point vs baseline measurement for certain treatment.
[2]: p-value for test whether there is a statistically significant difference between changes from baseline comparing to placebo.
All the p-values and estimates are derived from a linear mixed effect model with number of voids as dependent variables, treatments (placebo, 16000 ug, 24000 ug and total hMaxi-K), time point and interaction of time and treatment.
All doses = all hMaxi-K doses
SD = standard deviation;
SEM = standard error of the mean

TABLE 9
Number of Urge Incontinence episodes and Reduction Over Time - Efficacy Population

| | | hMaxi-K | | | | | | | |
|---|---|---|---|---|---|---|---|---|---|
| | | Placebo | | 16000 ug | | 24000 ug | | All Doses | |
| Visit | n | 4 | | 6 | | 3 | | 9 | |
| Visit 1A | Mean no. urge incontinence | 1.88 | (1.25) | 2.08 | (0.57) | 8.69 | (12.02) | 4.29 | (6.87) |
| (Screening) | episodes/24 hrs (SD) | | | | | | | | |
| Visit 2 | Mean no. urge incontinence | 1.82 | (1.52) | 1.91 | (0.83) | 3.81 | (3.30) | 2.54 | (2.01) |
| (Baseline) | episodes/24 hrs (SD) | | | | | | | | |
| Visit 3 | Mean no. urge incontinence | 1.43 | (1.32) | 1.29 | (1.08) | 2.74 | (0.25) | 1.77 | (1.13) |
| (Week 1) | episodes/24 hrs (SD) | | | | | | | | |
| | Mean change from baseline | −0.39 | (0.22) | −0.63 | (0.74) | −1.07 | (3.15) | −0.78 | (1.69) |
| | (SD) | | | | | | | | |
| | SEM | 0.11 | | 0.30 | | 1.82 | | 0.56 | |
| | P-value [1] | 0.460 | | 0.164 | | 0.103 | | 0.045 | |
| | P-value [2] | | | 0.718 | | 0.395 | | 0.470 | |
| | Difference of LS Means vs. | | | −0.24 | | −0.68 | | −0.46 | |
| | placebo | | | | | | | | |
| | 95% CI | | | −1.75, 1.27 | | −2.47, 1.10 | | −1.89, 0.97 | |
| Visit 4 | Mean no. urge incontinence | 1.23 | (1.27) | 0.86 | (1.09) | 2.95 | (1.35) | 1.56 | (1.51) |
| (Week 2) | episodes/24 hrs (SD) | | | | | | | | |
| | Mean change from baseline | −0.58 | (0.81) | −1.05 | (1.39) | −0.86 | (2.60) | −0.99 | (1.70) |
| | (SD) | | | | | | | | |
| | SEM | 0.40 | | 0.57 | | 1.50 | | 0.57 | |
| | P-value [1] | 0.277 | | 0.035 | | 0.177 | | 0.029 | |
| | P-value [2] | | | 0.487 | | 0.728 | | 0.559 | |
| | Difference of LS Means vs. | | | −0.47 | | −0.27 | | −0.37 | |
| | placebo | | | | | | | | |
| | 95% CI | | | −1.98, 1.04 | | −2.06, 1.51 | | −1.80, 1.06 | |
| Visit 5 | Mean no. urge incontinence | 1.14 | (0.95) | 0.66 | (0.81) | 3.10 | (2.08) | 1.47 | (1.72) |
| (Week 4) | episodes/24 hrs (SD) | | | | | | | | |
| | Mean change from baseline | −0.67 | (0.98) | −1.25 | (1.16) | −0.71 | (1.76) | −1.07 | (1.30) |
| | (SD) | | | | | | | | |
| | SEM | 0.49 | | 0.48 | | 1.01 | | 0.43 | |
| | P-value [1] | 0.216 | | 0.017 | | 0.251 | | 0.026 | |
| | P-value [2] | | | 0.393 | | 0.958 | | 0.623 | |
| | Difference of LS Means vs. | | | −0.58 | | −0.04 | | −0.31 | |
| | placebo | | | | | | | | |
| | 95% CI | | | −2.09, 0.93 | | −1.83, 1.74 | | −1.74, 1.12 | |
| Visit 6 | Mean no. urge incontinence | 1.02 | (1.15) | 0.50 | (0.92) | 2.57 | (2.13) | 1.19 | (1.66) |
| (Week 8) | episodes/24 hrs (SD) | | | | | | | | |
| | Mean change from baseline | −0.79 | (0.49) | −1.41 | (1.21) | −1.24 | (1.67) | −1.35 | (1.27) |
| | (SD) | | | | | | | | |
| | SEM | 0.25 | | 0.49 | | 0.97 | | 0.42 | |
| | P-value [1] | 0.153 | | 0.010 | | 0.067 | | 0.007 | |
| | P-value [2] | | | 0.363 | | 0.573 | | 0.407 | |
| | Difference of LS Means vs. | | | −0.62 | | −0.45 | | −0.53 | |
| | placebo | | | | | | | | |
| | 95% CI | | | −2.13, 0.89 | | −2.23, 1.34 | | −1.97, 0.90 | |
| Visit 7 | Mean no. urge incontinence | 1.25 | (1.09) | 0.64 | (0.75) | 3.29 | (2.27) | 1.52 | (1.84) |
| (Week 12) | episodes/24 hrs (SD) | | | | | | | | |
| | Mean change from baseline | −0.57 | (0.71) | −1.27 | (1.17) | −0.52 | (1.57) | −1.02 | (1.27) |
| | (SD) | | | | | | | | |
| | SEM | 0.35 | | 0.48 | | 0.90 | | 0.42 | |
| | P-value [1] | 0.290 | | 0.016 | | 0.389 | | 0.037 | |
| | P-value [2] | | | 0.306 | | 0.958 | | 0.601 | |
| | Difference of LS Means vs. | | | −0.70 | | 0.04 | | −0.33 | |
| | placebo | | | | | | | | |
| | 95% CI | | | −2.21, 0.81 | | −1.74, 1.83 | | −1.76, 1.10 | |
| Visit 8 (Exit | Mean no. urge incontinence | 0.86 | (0.76) | 0.62 | (0.84) | 1.52 | (1.39) | 0.92 | (1.06) |
| Visit) (Week 24) | episodes/24 hrs (SD) | | | | | | | | |
| | Mean change from baseline | −0.96 | (0.94) | −1.29 | (1.10) | −2.29 | (2.72) | −1.62 | (1.69) |
| | (SD) | | | | | | | | |

| | | | | |
|---|---|---|---|---|
| SEM | 0.47 | 0.45 | 1.57 | 0.56 |
| P-value [1] | 0.094 | 0.015 | 0.005 | 0.001 |
| P-value [2] | | 0.616 | 0.122 | 0.212 |
| Difference of LS Means vs. | | −0.34 | −1.33 | −0.83 |
| placebo | | | | |
| 95% CI | | −1.84, 1.17 | −3.11, 0.46 | −2.26, 0.60 |

[1]: P-value to test whether there is a statistically significant difference between values measured at certain time point vs baseline measurement for certain treatment.
[2]: P-value for test whether there is a statistically significant difference between changes from baseline comparing to placebo.
All the P-values and estimates are derived from a linear mixed effect model with number of voids as dependent variables, treatments (placebo, 16000 ug, 24000 ug and total hMaxi-K), time point and interaction of time and treatment.
All doses = all hMaxi-K doses
SD = standard deviation;
SEM = standard error of the mean

TABLE 10
Participant Perception of Response to Treatment - Efficacy Population

| | | Placebo, n (%) | hMaxi-K, n (%) | | |
|---|---|---|---|---|---|
| | | Placebo | 16000 ug | 24000 ug | All Doses |
| V3 (N = 13) | No benefit | 3 (75.00) | 1 (16.67) | 0 | 1 (11.11) |
| | Yes, a little benefit | 1 (25.00) | 1 (16.67) | 3 (100.0) | 4 (44.44) |
| | Yes, very much | 0 | 4 (66.67) | 0 | 4 (44.44) |
| | benefit | | | | |
| | P-value | | 0.1429 | 0.1429 | 0.0190 |
| V4 (N = 13) | No benefit | 3 (75.00) | 1 (16.67) | 0 | 1 (11.11) |
| | Yes, a little benefit | 1 (25.00) | 1 (16.67) | 2 (66.67) | 3 (33.33) |
| | Yes, very much | 0 | 4 (66.67) | 1 (33.33) | 5 (55.56) |
| | benefit | | | | |
| | P-value | | 0.1429 | 0.2286 | 0.1202 |
| V5 (N = 13) | No benefit | 3 (75.00) | 1 (16.67) | 0 | 1 (11.11) |
| | Yes, a little benefit | 1 (25.00) | 0 | 2 (66.67) | 2 (22.22) |
| | Yes, very much | 0 | 5 (83.33) | 1 (33.33) | 6 (66.67) |
| | benefit | | | | |
| | P-value | | 0.0238 | 0.2286 | 0.0126 |
| V6 (N = 13) | No benefit | 3 (75.00) | 1 (16.67) | 0 | 1 (11.11) |
| | Yes, a little benefit | 1 (25.00) | 2 (33.33) | 2 (66.67) | 4 (44.44) |
| | Yes, very much | 0 | 3 (50.00) | 1 (33.33) | 4 (44.44) |
| | benefit | | | | |
| | P-value | | 0.2286 | 0.2286 | 0.2727 |
| V7 (N = 13) | No benefit | 3 (75.00) | 2 (33.33) | 0 | 2 (22.22) |
| | Yes, a little benefit | 1 (25.00) | 1 (16.67) | 2 (66.67) | 3 (33.33) |
| | Yes, very much | 0 | 3 (50.00) | 1 (33.33) | 4 (44.44) |
| | benefit | | | | |
| | P-value | | 0.2857 | 0.2286 | 0.2727 |
| V8 (N = 13) | No benefit | 3 (75.00) | 2 (33.33) | 0 | 2 (22.22) |
| | Yes, a little benefit | 1 (25.00) | 1 (16.67) | 2 (66.67) | 3 (33.33) |
| | Yes, very much | 0 | 3 (50.00) | 1 (33.33) | 4 (44.44) |
| | benefit | | | | |
| | P-value | | 0.2857 | 0.2286 | 0.2727 |

Note:
p-values are nominal and for chi-square test to see whether perception of response to treatment are different for patients received treatment and those received placebo.
All doses = all hMaxi-K doses

TABLE 11
Change in the Mean Number of Urge Incontinence Episode per 24 Hours - Efficacy Population

| | | hMaxi-K | | | |
|---|---|---|---|---|---|
| | | Placebo | 16000 ug | 24000 ug | All Doses |
| Visit | n | 4 | 6 | 3 | 9 |

Urge incontinence
episode per 24
hours

| Visit 1A | Mean (SD) | 1.88 | (1.25) | 2.08 | (0.57) | 8.69 | (12.02) | 4.29 | (6.87) |
|---|---|---|---|---|---|---|---|---|---|
| Visit 2 | Mean (SD) | 1.82 | (1.52) | 1.91 | (0.83) | 3.81 | (3.30) | 2.54 | (2.01) |
| Visit 3 | Mean (SD) | 1.43 | (1.32) | 1.29 | (1.08) | 2.74 | (0.25) | 1.77 | (1.13) |
| Visit 4 | Mean (SD) | 1.23 | (1.27) | 0.86 | (1.09) | 2.95 | (1.35) | 1.56 | (1.51) |
| Visit 5 | Mean (SD) | 1.14 | (0.95) | 0.66 | (0.81) | 3.10 | (2.08) | 1.47 | (1.72) |
| Visit 6 | Mean (SD) | 1.02 | (1.15) | 0.50 | (0.92) | 2.57 | (2.13) | 1.19 | (1.66) |
| Visit 7 | Mean (SD) | 1.25 | (1.09) | 0.64 | (0.75) | 3.29 | (2.27) | 1.52 | (1.84) |
| Visit 8 | Mean (SD) | 0.86 | (0.76) | 0.62 | (0.84) | 1.52 | (1.39) | 0.92 | (1.06) |
| (Exit Visit) | | | | | | | | | |
| Change from | | | | | | | | | |
| Baseline V2 | | | | | | | | | |
| Visit 3 | Mean (SD) | −0.39 | (0.22) | −0.63 | (0.74) | −1.07 | (3.15) | −0.78 | (1.69) |
| | p-value [1] | 0.460 | | 0.164 | | 0.103 | | 0.045 | |
| | p-value [2] | | | 0.718 | | 0.395 | | 0.470 | |
| | Difference of LS | | | −0.24 | | −0.68 | | −0.46 | |
| | Means vs. placebo | | | | | | | | |
| | 95% CI | | | −1.75, 1.27 | | −2.47, 1.10 | | −1.89, 0.97 | |
| | p-value [3] | | | | | 0.545 | | | |
| | Difference of LS | | | | | −0.44 | | | |
| | Means 24000 ug vs. | | | | | | | | |
| | 16000 ug | | | | | | | | |
| | 95% CI | | | | | −2.10, 1.21 | | | |
| Visit 4 | Mean (SD) | −0.58 | (0.81) | −1.05 | (1.39) | −0.86 | (2.60) | −0.99 | (1.70) |
| | p-value [1] | 0.277 | | 0.035 | | 0.177 | | 0.029 | |
| | p-value [2] | | | 0.487 | | 0.728 | | 0.559 | |
| | Difference of LS | | | −0.47 | | −0.27 | | −0.37 | |
| | Means vs. placebo | | | | | | | | |
| | 95% CI | | | −1.98, 1.04 | | −2.06, 1.51 | | −1.80, 1.06 | |
| | p-value [3] | | | | | 0.789 | | | |
| | Difference of LS | | | | | 0.19 | | | |
| | Means 24000 ug vs. | | | | | | | | |
| | 16000 ug | | | | | | | | |
| | 95% CI | | | | | −1.46, 1.85 | | | |
| Visit 5 | Mean (SD) | −0.67 | (0.98) | −1.25 | (1.16) | −0.71 | (1.76) | −1.07 | (1.30) |
| | p-value [1] | 0.216 | | 0.017 | | 0.251 | | 0.026 | |
| | p-value [2] | | | 0.393 | | 0.958 | | 0.623 | |
| | Difference of LS | | | −0.58 | | −0.04 | | −0.31 | |
| | Means vs. placebo | | | | | | | | |
| | 95% CI | | | −2.09, 0.93 | | −1.83, 1.74 | | −1.74, 1.12 | |
| | p-value [3] | | | | | 0.465 | | | |
| | Difference of LS | | | | | 0.54 | | | |
| | Means 24000 ug vs. | | | | | | | | |
| | 16000 ug | | | | | | | | |
| | 95% CI | | | | | −1.11, 2.19 | | | |
| Visit 6 | Mean (SD) | −0.79 | (0.49) | −1.41 | (1.21) | −1.24 | (1.67) | −1.35 | (1.27) |
| | p-value [1] | 0.153 | | 0.010 | | 0.067 | | 0.007 | |
| | p-value [2] | | | 0.363 | | 0.573 | | 0.407 | |
| | Difference of LS | | | −0.62 | | −0.45 | | −0.53 | |
| | Means vs. placebo | | | | | | | | |
| | 95% CI | | | −2.13, 0.89 | | −2.23, 1.34 | | −1.97, 0.90 | |
| | p-value [3] | | | | | 0.810 | | | |
| | Difference of LS | | | | | 0.17 | | | |
| | Means 24000 ug vs. | | | | | | | | |
| | 16000 ug | | | | | | | | |
| | 95% CI | | | | | −1.48, 1.83 | | | |
| Visit 7 | Mean (SD) | −0.57 | (0.71) | −1.27 | (1.17) | −0.52 | (1.57) | −1.02 | (1.27) |
| | p-value [1] | 0.290 | | 0.016 | | 0.389 | | 0.037 | |
| | p-value [2] | | | 0.306 | | 0.958 | | 0.601 | |
| | Difference of LS | | | −0.70 | | 0.04 | | −0.33 | |
| | Means vs. placebo | | | | | | | | |
| | 95% CI | | | −2.21, 0.81 | | −1.74, 1.83 | | −1.76, 1.10 | |
| | p-value [3] | | | | | 0.321 | | | |
| | Difference of LS | | | | | 0.75 | | | |
| | Means 24000 ug vs. | | | | | | | | |
| | 16000 ug | | | | | | | | |
| | 95% CI | | | | | −0.91, 2.40 | | | |
| Visit 8 | Mean (SD) | −0.96 | (0.94) | −1.29 | (1.10) | −2.29 | (2.72) | −1.62 | (1.69) |

| | | | | | |
|---|---|---|---|---|---|
| (Exit Visit) | p-value [1] | 0.094 | 0.015 | 0.005 | 0.001 |
| | p-value [2] | | 0.616 | 0.122 | 0.212 |
| | Difference of LS | | −0.34 | −1.33 | −0.83 |
| | Means vs. placebo | | | | |
| | 95% CI | | −1.84, 1.17 | −3.11, 0.46 | −2.26, 0.60 |
| | p-value [3] | | | 0.199 | |
| | Difference of LS | | | −0.99 | |
| | Means 24000 ug vs. | | | | |
| | 16000 ug | | | | |
| | 95% CI | | | −2.65, 0.66 | |

[1]: p-value to test whether there is a statistically significant difference between values measured at certain time point vs baseline measurement for certain treatment.
[2]: p-value for test whether there is a statistically significant difference between changes from baseline comparing to placebo.
[3]: p-value for test whether there is difference between 24000 ug group vs. 16000 ug group. ssAll the p-values and estimates are derived from a linear mixed effect model with number of urge incontinence episode per 24 hours as dependent variables, treatments (placebo, 16000 ug, 24000 ug and total hMaxi-K), time point and interaction of time and treatment.

TABLE 12
Change in the Weight (gm) of 72 Hour Pad Test - Safety Population

| | | hMaxi-K | | | | | | | |
|---|---|---|---|---|---|---|---|---|---|
| | | Placebo | | 16000 ug | | 24000 ug | | All Doses | |
| Visit | n | 4 | | 6 | | 3 | | 9 | |
| Visit 1A | Mean (SD) weight of | 29.33 | (20.03) | 345.00 | (726.50) | 611.67 | (703.53) | 433.89 | (686.58) |
| Screening | 72 hr. pad test | | | | | | | | |
| Visit 2 | Mean (SD) weight of | 259.25 | (417.95) | 314.00 | (663.23) | 677.33 | (643.96) | 435.11 | (641.56) |
| Baseline | 72 hr. pad test | | | | | | | | |
| Visit 3 | Mean (SD) weight of | 133.50 | (206.99) | 241.67 | (541.39) | 518.03 | (499.37) | 333.79 | (514.42) |
| (Week 1) | 72 hr. pad test | | | | | | | | |
| | Mean (SD) change from | −125.75 | (211.14) | −72.33 | (123.08) | −159.30 | (144.90) | −101.32 | (128.87) |
| | baseline in pad weight | | | | | | | | |
| | P-value [1] | 0.044 | | 0.127 | | 0.024 | | 0.013 | |
| | P-value [2] | | | 0.446 | | 0.598 | | 0.937 | |
| | Difference of LS Means | | | 53.42 | | −43.14 | | 5.14 | |
| | vs. placebo | | | | | | | | |
| | 95% CI | | | −102.87, 209.70 | | −228.08, 141.80 | | −143.13, 153.41 | |
| Visit 4 | Mean (SD) weight of | 119.00 | (177.72) | 231.83 | (509.77) | 528.00 | (501.86) | 330.56 | (497.30) |
| (Week 2) | 72 hr. pad test | | | | | | | | |
| | Mean (SD) change from | −140.25 | (242.66) | −82.17 | (155.66) | −149.33 | (142.12) | −104.56 | (146.02) |
| | baseline in pad weight | | | | | | | | |
| | P-value [1] | 0.029 | | 0.090 | | 0.031 | | 0.013 | |
| | P-value [2] | | | 0.409 | | 0.818 | | 0.762 | |
| | Difference of LS Means | | | 58.08 | | −18.67 | | 19.71 | |
| | vs. placebo | | | | | | | | |
| | 95% CI | | | −98.20, 214.37 | | −203.61, 166.27 | | −128.57, 167.98 | |
| Visit 5 | Mean (SD) weight of | 100.75 | (84.24) | 212.00 | (485.13) | 494.67 | (508.22) | 306.22 | (481.29) |
| (Week 4) | 72 hr. pad test | | | | | | | | |
| | Mean (SD) change from | −158.50 | (345.31) | −102.00 | (179.22) | −182.67 | (153.16) | −128.89 | (166.03) |
| | baseline in pad weight | | | | | | | | |
| | P-value [1] | 0.017 | | 0.045 | | 0.014 | | 0.005 | |
| | P-value [2] | | | 0.421 | | 0.679 | | 0.861 | |
| | Difference of LS Means | | | 56.50 | | −33.76 | | 11.37 | |
| | vs. placebo | | | | | | | | |
| | 95% CI | | | −99.79, 212.79 | | −218.69, 151.18 | | −136.90, 159.64 | |
| Visit 6 [3] | Mean (SD) weight of | 164.00 | (272.19) | 186.33 | (427.25) | 489.33 | (425.48) | 287.33 | (426.96) |
| Week 8) | 72 hr. pad test | | | | | | | | |
| | Mean (SD) change from | −95.25 | (145.96) | −127.67 | (236.90) | −188.00 | (361.87) | −147.78 | (262.15) |
| | baseline in pad weight | | | | | | | | |
| | P-value [1] | 0.105 | | 0.018 | | 0.012 | | 0.003 | |
| | P-value [2] | | | 0.639 | | 0.232 | | 0.318 | |
| | Difference of LS Means | | | −32.42 | | −102.34 | | −67.38 | |
| | vs. placebo | | | | | | | | |
| | 95% CI | | | −188.70, 123.87 | | −287.28, 82.60 | | −215.65, 80.89 | |
| Visit 7 [3] | Mean (SD) weight of | 177.50 | (307.75) | 307.50 | (709.54) | 545.3 | (621.50) | 386.78 | (652.19) |
| (Week 12) | 72 hr. pad test | | | | | | | | |
| | Mean (SD) change from | −81.75 | (110.34) | −6.50 | (52.31) | −191.00 | (159.81) | −52.63 | (113.57) |
| | baseline in pad weight | | | | | | | | |
| | P-value [1] | 0.154 | | 0.881 | | 0.224 | | 0.256 | |
| | P-value [2] | | | 0.292 | | 0.860 | | 0.671 | |
| | Difference of LS Means | | | 75.25 | | −16.46 | | 29.40 | |
| | vs. placebo | | | | | | | | |
| | 95% CI | | | −81.04, 231.54 | | −228.54, 195.63 | | −127.70, 186.49 | |
| Visit 8 [3] | Mean (SD) weight of | 85.00 | (126.10) | 225.00 | (520.04) | 596.67 | (528.52) | 348.89 | (522.87) |
| (Week 24) | 72 hr. pad test | | | | | | | | |
| | Mean (SD) change from | −174.25 | (293.32) | −89.00 | (145.01) | −80.67 | (189.03) | −86.22 | (148.64) |
| | baseline in pad weight | | | | | | | | |

| | | | | |
|---|---|---|---|---|
| P-value [1] | 0.011 | 0.071 | 0.171 | 0.042 |
| P-value [2] | | 0.238 | 0.318 | 0.219 |
| Difference of LS Means | | 85.25 | 83.99 | 84.62 |
| vs. placebo | | | | |
| 95% CI | | −71.04, 241.54 | −100.94, 268.93 | −63.65, 232.89 |

[1]: P-value to test whether there is a statistically significant difference between values measured at certain time point vs baseline measurement for certain treatment.
[2]: P-value for test whether there is a statistically significant difference between changes from baseline comparing to placebo.
[3]: Results include a value of 0 for subject 002019 whose results were incorrectly entered into the database. Results verified by site and CRA.
All the P-values and estimates are derived from a linear mixed effect model with weight of 72-hour pad test as dependent variables, treatments (placebo, 16000 ug, 24000 ug and total hMaxi-K), time point and interaction of time and treatment.
All doses = all hMaxi-K doses
SD = standard deviation

1. A method of treating or alleviating a sign or symptom of overactive bladder syndrome or detrusor overactivity in a human subject comprising administering intradetrusorally to at least two or more sites a unit dose of a composition comprising a vector having a promoter and a nucleic acid encoding a Maxi-K channel peptide.

2. The method of claim 1, wherein the promoter is a smooth muscle promoter.

3. The method of claim 1, wherein the promoter is a cytomegalovirus intermediate-early promoter.

4. The method of claim 1, wherein the unit dose is a single unit dose.

5. The method of claim 1, wherein the unit dose is between about 5.000-50,000 mcg.

6. (canceled)

7. The method of claim 1, wherein the unit dose is 16,000 mcg or 24,000 mcg.

8. The method of claim 1, wherein the composition is administered at 5 or more sites.

9-11. (canceled)

12. The method of claim 1 wherein the sign or symptom is frequency of micturition or urgency.

13. The method of claim 1, wherein the vector comprises nucleic acid elements in the following order:
a. a human cytomegalovirus intermediate-early promoter sequence;
b. a T7 priming site sequence;
c. a hSlo open reading frame sequence;
d. a BGH polyadenylation signal sequence,
e. a kanamycin resistance sequence; and
f. a pUC origin of replication sequence.

14. The method of claim 13, wherein the human cytomegalovirus intermediate-early promoter sequence is SEQ II) NO: 1.

15. The method of claim 13, wherein T7 priming site sequence is SEQ ID NO: 2.

16. The method of claim 13, wherein the BGH polyadenylation signal sequence is SEQ ID NO: 3.

17. The method of claim 13, wherein the kanamycin resistance sequence is SEQ ID NO: 5.

18. The method of claim 13, wherein the a pUC origin of replication sequence is SEQ ID NO: 4.

19. The method of claim 13, wherein the hSlo open reading frame sequence is SEQ ID NO. 7.

20. The method of claim 19, wherein the hSlo open reading frame sequence has a single point mutation at nucleotide position 1054 of SEQ ID NO: 7, and wherein said point mutation results in serine at position 352 of SEQ ID NO: 8.

21. A vector comprising nucleic acid elements in the following order:
g. a human cytomegalovirus intermediate-early promoter sequence comprising SEQ ID NO. 1;
h. a T7 priming site sequence comprising SEQ ID NO: 2;
i. a hSlo open reading frame sequence comprising SEQ ID NO: 7;
j. a BGH polyadenylation signal sequence comprising SEQ ID NO: 3;
k. a kanamycin resistance sequence comprising SEQ ID NO: 5; and
l. a pUC origin of replication sequence comprising SEQ ID NO: 4.

22-24. (canceled)

25. A pharmaceutical composition, comprising a plurality of the vector of claim 23 and a pharmaceutically acceptable diluent or carrier.

26. The pharmaceutical composition of claim 25, wherein the pharmaceutical composition is formulated for injection into smooth muscle.

27. The pharmaceutical composition of claim 25, wherein the plurality of the vector is combined with a 20-25% sucrose in saline solution.

28-31. (canceled)


SEQUENCE LISTING (11 sequences)

SEQ ID NO: 1 - DNA, 508 bp, human cytomegalovirus
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 2 - DNA, 20 bp, Artificial Sequence
  T7 priming site
taatacgactcactataggg

SEQ ID NO: 3 - DNA, 239 bp, Artificial Sequence
  BGH polyA
agcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctagtgggctct

SEQ ID NO: 4 - DNA, 679 bp, Artificial Sequence
  pUC origin of replication
ccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 5 - DNA, 800 bp, Artificial Sequence
  kanamycin resistance marker
ttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 6 - DNA, 3779 bp, homo sapiens
atggcaaacggtggcggcggcggcggcggcagcagcggcggcggcggcggcggcggcggaggcagcggtcttagaatgagcagcaatatccacgcgaaccatctcagcctagacgcgtcctcctcctcctcctcctcctcttcctcttcttcttcttcctcctcctcttcctcctcgtcctcggtccacgagcccaagatggatgcgctcatcatcccggtgaccatggaggtgccgtgcgacagccggggccaacgcatgtggtgggctttcctggcctcctccatggtgactttcttcgggggcctcttcatcatcttgctctggcggacgctcaagtacctgtggaccgtgtgctgccactgcgggggcaagacgaaggaggcccagaagattaacaatggctcaagccaggcggatggcactctcaaaccagtggatgaaaaagaggaggcagtggccgccgaggtcggctggatgacctccgtgaaggactgggcgggggtgatgatatccgcccagacactgactggcagagtcctggttgtcttagtctttgctctcagcatcggtgcacttgtaatatacttcatagattcatcaaacccaatagaatcctgccagaatttctacaaagatttcacattacagatcgacatggctttcaacgtgttcttccttctctactttggcttgcggtttattgcagccaacgataaattgtggttctggctggaagtgaactctgtagtggatttcttcacggtgccccccgtgtttgtgtctgtgtacttaaacagaagttggcttggtttgagatttttaagagctctgagactgatacagttttcagaaattttgcagtttctgaatattcttaaaacaagtaattccatcaagctggtgaatctgctctccatatttatcagcacgtggctgactgcagctgggttcatccatttggtggagaattcaggggacccatgggaaaatttccaaaacaaccaggctctcacctactgggaatgtgtcatttactcatggtcacaatgtccaccgttggttatggggatgtttatgcaaaaaccacacttcggcgcctcttcatggtcttcttcatcctcgggggactggccatgtttgccagctacgtccctgaaatcatagagttaataggaaaccgcaagaaatacgggggctcctatagtgcggttagtggaagaaagcacattgtggtctgcggacacatcactctggagagtgtttccaacttcctgaaggactttctgcacaaggaccgggatgacgtcaatgtggagatcgtttttcttcacaacatctcccccaacctggagcttgaagctctgttcaaacgacattttactcaggtggaattttatcagggttccgtcctcaatccacatgatcttgcaagagtcaagatagagtcagcagatgcatgcctgatccttgccaacaagtactgcgctgacccggatgcggaggatgcctcgaatatcatgagagtaatctccataaagaactaccatccgaagataagaatcatcactcaaatgctgcagtatcacaacaaggcccatctgctaaacatccgagctggaattggaaagaaggtgatgacgcaatctgcctcgcagagttgaagttgggcttcatagcccagagctgcctggctcaaggcctctccaccatgcttgccaaccttctccatgaggtcattcataaagattgaggaagacacatggcagaaatactacttggaaggagtctcaaatcaaatgtacacagaatatctctccagtgccttcgtgggtctgtccttccctactgtttgtgagctgtgttttgtgaagctcaagctcctaatgatagccattgagtacaagtctgccaaccgagagagccgtatattaattaatcctggaaaccattttaagatccaagaaggtactttaggatttttcatcgcaagtgatgccaaagaagttaaaagggcatttttttactgcaaggcctgtcatgatgacatcacagatcccaaaagaataaaaaaatgtggctgcaaacggcttgaagatgagcagccgtcaacactatcaccaaaaaaaaagcaacggaatggaggcatgcggaactcacccaacacctcgcctaagctgatgaggcatgaccccttgttaattcctggcaatgatcagattgacaacatggactccaatgtgaagaagtacgactctactgggatgtttcactggtgtgcacccaaggagatagagaaagtcatcctgactcgaagtgaagctgccatgaccgtcctgagtggccatgtcgtggtctgcatctttggcgacgtcagctcagccctgatcggcctccggaacctggtgatgccgctccgtgccagcaactttcattaccatgagctcaagcacattgtgtttgtgggctctattgagtacctcaagcgggaatgggagacgcttcataacttccccaaagtgtccatattgcctggtacgccattaagtcgggctgatttaagggctgtcaacatcaacctctgtgacatgtgcgttatcctgtcagccaatcagaataatattgatgatacttcgctgcaggacaaggaatgcatcttggcgtcactcaacatcaaatctatgcagtttgatgacagcatcggagtcttgcaggctaattcccaagggttcacacctccaggaatggatagatcctctccagataacagcccagtgcacgggatgttacgtcaaccatccatcacaactggggtcaacatccccatcatcactgaactagtgaacgatactaatgttcagtttttggaccaagacgatgatgatgaccctgatacagaactgtacctcacgcagccctttgcctgtgggacagcatttgccgtcagtgtcctggactcactcatgagcgcgacgtacttcaatgacaatatcctcaccctgatacggaccctggtgaccggaggagccacgccggagctggaggctctgattgctgaggaaaacgcccttagaggtggctacagcaccccgcagacactggccaatagggaccgctgccgcgtggcccagttagctctgctcgatgggccatttgcggacttaggggatggtggttgttatggtgatctgttctgcaaagctctgaaaacatataatatgctttgttttggaatttaccggctgagagatgctcacctcagcacccccagtcagtgcacaaagaggtatgtcatcaccaacccgccctatgagtttgagctcgtgccgacggacctgatcttctgcttaatgcagtttgaccacaatgccggccagtcccgggccagcctgtcccattcctcccactcgtcgcagtcctccagcaagaagagctcctctgttcactccatcccatccacagcaaaccgacagaaccggcccaagtccagggagtcccgggacaaacagaagtacgtgcaggaagagcggctttgatatgtgtatccaccgccactgtgtgaaactgtatctgccactcatttccccagttggtgtttccaacaaagtaactttccctgttttcccctgtagtccccccctttttttttacacatatttgcatatgtatgatagtgtgcatgtggttgtcatttttatttcaccaccataaaacccttgagcacaacagcaaataagcagacgggctccggaattcctgcagcccgggggatccactag

SEQ ID NO: 7 - DNA, 3534 bp, Homo sapiens
atggcaaatggtggcggcggcggcggcggcagcagcggcggcggcggcggcggcggaggcagcagtcttagaatgagtagcaatatccacgcgaaccatctcagcctagacgtgtcctcctcctcctcctcctcctcttcctcttcttcttcttcctcctcctcttcctcctcgtcctcggtccacgagcccaagatggatgcgctcatcatcccggtgaccatggaggtgccgtgcgacagccggggccaacgcatgtggtgggctttcctggcctcctccatggtgactttcttcgggggcctcttcatcatcttgctctggcggacgctcaagtacctgtggaccgtgtgctgccactgcgggggcaagacgaaggaggcccagaagattaacaatggctcaagccaggcggatggcactctcaaaccagtggatgaaaaagaggaggcagtggccgccgaggtcggctggatgacctccgtgaaggactgggcgggggtgatgatatccgcccagacactgactggcagagtcctggttgtcttagtctttgctctcagcatcggtgcacttgtaatatacttcatagattcatcaaacccaatagaatcctgccagaatttctacaaagatttcacattacagatcgacatggctttcaacgtgttcttccttctctacttcggcttgcggtttattgcagccaacgataaattgtggttctggctggaagtgaactctgtagtggatttcttcacggtgccccccgtgtttgtgtctgtgtacttaaacagaagttggcttggtttgagatttttaagagctctgagactgatacagttttcagaaattttgcagtttctgaatattcttaaaacaagtaattccatcaagctggtgaatctgctctccatatttatcagcacgtggctgactgcagccgggttcatccatttggtggagaattcaggggacccatgggaaaatttccaaaacaaccaggctctcacctactgggaatgtgtctatttactcatggtcacaatgtccaccgttggttatggggatgtttatgcaaaaaccacacttgggcgcctcttcatggtcttcttcatcctcgggggactggccatgtttgccagctacgtccctgaaatcatagagttaataggaaaccgcaagaaatacgggggctcctatagtgcggttagtggaagaaagcacattgtggtctgcggacacatcactctggagagtgtttccaacttcctgaaggactttctgcacaaggaccgggatgacgtcaatgtggagatcgtttttcttcacaacatctcccccaacctggagcttgaagctctgttcaaacgacattttactcaggtggaattttatcagggttccgtcctcaatccacatgatcttgcaagagtcaagatagagtcagcagatgcatgcctgatccttgccaacaagtactgcgctgacccggatgcggaggatgcctcgaatatcatgagagtaatctccataaagaactaccatccgaagataagaatcatcactcaaatgctgcagtatcacaacaaggcccatctgctaaacatcccgagctggaattggaaagaaggtgatgacgcaatctgcctcgcagagttgaagttgggcttcatagcccagagctgcctggctcaaggcctctccaccatgcttgccaacctcttctccatgaggtcattcataaagattgaggaagacacatggcagaaatactacttggaaggagtctcaaatgaaatgtacacagaatatctctccagtgccttcgtgggtctgtccttccctactgtttgtgagctgtgttttgtgaagctcaagctcctaatgatagccattgagtacaagtctgccaaccgagagagccgtatattaattaatcctggaaaccatcttaagatccaagaaggtactttaggatttttcatcgcaagtgatgccaaagaagttaaaagggcatttttttactgcaaggcctgtcatgatgacatcacagatcccaaaagaataaaaaaatgtggctgcaaacggcttgaagatgagcagccgtcaacactatcaccaaaaaaaaagcaacggaatggaggcatgcggaactcacccaacacctcgcctaagctgatgaggcatgaccccttgttaattcctggcaatgatcagattgacaacatggactccaatgtgaagaagtacgactctactgggatgtttcactggtgtgcacccaaggagatagagaaagtcatcctgactcgaagtgaagctgccatgaccgtcctgagtggccatgtcgtggtctgcatctttggcgacgtcagctcagccctgatcggcctccggaacctggtgatgccgctccgtgccagcaactttcattaccatgagctcaagcacattgtgtttgtgggctctattgagtacctcaagcgggaatgggagacgcttcataacttccccaaagtgtccatattgcctggtacgccattaagtcgggctgatttaagggctgtcaacatcaacctctgtgacatgtgcgttatcctgtcagccaatcagaataatattgatgatacttcgctgcaggacaaggaatgcatcttggcgtcactcaacatcaaatctatgcagtttgatgacagcatcggagtcttgcaggctaattcccaagggttcacacctccaggaatggatagatcctctccagataacagcccagtgcacgggatgttacgtcaaccatccatcacaactggggtcaacatccccatcatcactgaactagtgaacgatactaatgttcagtttttggaccaagacgatgatgatgaccctgatacagaactgtacctcacgcagccctttgcctgtgggacagcatttgccgtcagtgtcctggactcactcatgagcgcgacgtacttcaatgacaatatcctcaccctgatacggaccctggtgaccggaggagccacgccggagctggaggctctgattgctgaggaaaacgcccttagaggtggctacagcaccccgcagacactggccaatagggaccgctgccgcgtggcccagttagctctgctcgatgggccatttgcggacttaggggatggtggttgttatggtgatctgttctgcaaagctctgaaaacatataatatgctttgttttggaatttaccggctgagagatgctcacctcagcacccccagtcagtgcacaaagaggtatgtcatcaccaacccgccctatgagtttgagctcgtgccgacggacctgatcttctgcttaatgcagtttgaccacaatgccggccagtcccgggccagcctgtcccattcctcccactcgtcgcagtcctccagcaagaagagctcctctgttcactccatcccatccacagcaaaccgacagaaccggcccaagtccagggagtcccgggacaaacagaagtacgtgcaggaagagcggctt

SEQ ID NO: 8 - protein, 1178 aa, homo sapiens
MANGGGGGGGSSGGGGGGGGSSLRMSSNIHANHLSLDVSSSSSSSSSSSSSSSSSSSSSSVHEPKMDALIIPVTMEVPCDSRGQRMWWAFLASSMVTFFGGLFIILLWRTLKYLWTVCCHCGGKTKEAQKINNGSSQADGTLKPVDEKEEAVAAEVGWMTSVKDWAGVMISAQTLTGRVLVVLVFALSIGALVIYFIDSSNPIESCQNFYKDFTLQIDMAFNVFFLLYFGLRFIAANDKLWFWLEVNSVVDFFTVPPVFVSVYLNRSWLGLRFLRALRLIQFSEILQFLNILKTSNSIKLVNLLSIFISTWLTAAGFIHLVENSGDPWENFQNNQALTYWECVYLLMVTMSTVGYGDVYAKTTLGRLFMVFFILGGLAMFASYVPEIIELIGNRKKYGGSYSAVSGRKHIVVCGHITLESVSNFLKDFLHKDRDDVNVEIVFLHNISPNLELEALFKRHFTQVEFYQGSVLNPHDLARVKIESADACLILANKYCADPDAEDASNIMRVISIKNYHPKIRIITQMLQYHNKAHLLNIPSWNWKEGDDAICLAELKLGFIAQSCLAQGLSTMLANLFSMRSFIKIEEDTWQKYYLEGVSNEMYTEYLSSAFVGLSFPTVCELCFVKLKLLMIAIEYKSANRESRILINPGNHLKIQEGTLGFFIASDAKEVKRAFFYCKACHDDITDPKRIKKCGCKRLEDEQPSTLSPKKKQRNGGMRNSPNTSPKLMRHDPLLIPGNDQIDNMDSNVKKYDSTGMFHWCAPKEIEKVILTRSEAAMTVLSGHVVVCIFGDVSSALIGLRNLVMPLRASNFHYHELKHIVFVGSIEYLKREWETLHNFPKVSILPGTPLSRADLRAVNINLCDMCVILSANQNNIDDTSLQDKECILASLNIKSMQFDDSIGVLQANSQGFTPPGMDRSSPDNSPVHGMLRQPSITTGVNIPIITELVNDTNVQFLDQDDDDDPDTELYLTQPFACGTAFAVSVLDSLMSATYFNDNILTLIRTLVTGGATPELEALIAEENALRGGYSTPQTLANRDRCRVAQLALLDGPFADLGDGGCYGDLFCKALKTYNMLCFGIYRLRDAHLSTPSQCTKRYVITNPPYEFELVPTDLIFCLMQFDHNAGQSRASLSHSSHSSQSSSKKSSSVHSIPSTANRQNRPKSRESRDKQKYVQEERL

SEQ ID NO: 9 - DNA, 1419 bp, Artificial Sequence
  SM22alpha promoter sequence
gaattcaggacgtaatcagtggctggaaagcaagagctctagaggagctctgacccttccttcagatgccacaaggaggtgctggagttctatgcaccaacagccaggctggctgtagtggattgagcgtctgaggctgcacctctctgggttctgggtgagactgaccctgcctgagggttctctccttccctctctctccctctccctctccctctctctgtttcctgaggtttccaggattggggatgacaccactaaagccttaccttttaagaagttgcattcagtgagtgtgtgcagataggggcagaggagagctggttctgtctccactgtgtttggtcttgtcagaccatcaggtgtgatagcagttgtctttaaccctaaccctgagccttcccttcccaagaccactgaagctaggtgcaagataagtggggaccctttaggatctttcacgataaggactattttgaagggagggagggtgacactgtttaccctagtgtctccagccttgccaggccttaaacatccgcccattgtcaaggggccagggttgacttgctgctaaacaaggcactccctagagaagcagcataccatacctgtgggcaggatgacccatgttctgccacgcacttggtaggccactttgaacctcaattttctcaactgttaaatggggtggtaactgataaaggggaacgtgaaaggaaggcgtttgcatagtgcctggttgtgcaggtcaagactagttcccaccaactcgattttaaagccttgcaagaaggtggccttgcaggttcctttgtcgggccaaactctagaatgcctccccctttctagagcagacccaagtccgggtaacaaggaagggtttcagggtcctgcccattcccggccgccctcagcaccgccccgccccgacccccgcagcatctccacagcttattatagcttaaaccctgcagccaactcctttctgggactcagaagacatagcaggtactgaacgtctcacctgctgaggtggtcctagtcctcacccgctctagcccgctagaagccttggaactatctcataccaggctgcacttgtttgtcttctcattgataaaaggtttaagcatgcagagaatgtctccggctgcccccgacagactgctccaacttggtgtctttccccaaatatggagcctgtgtggagtgagtggggcggcccggggtggtgagccaagcagacttccatgggcagggaggggcgccagcggacggcagaggggtgacatcactgcctaggcggcctttaaacccctcacccagccggcgccccagcccgtctgccccagcccagacaccgaagctactctccttccagtccacaaacgaccaagccttgtaagtgcaagtcat

SEQ ID NO: 10 - DNA, 2999 bp, Artificial Sequence
  pVAX vector
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 11 - DNA, 33 bp, Artificial Sequence
  mutated Slo sequence
atggtcacaatgtcctccgttggttatggggat